(19) United States
(12) Patent Application Publication
(10) Pub. No.: US 2008/0021044 A1
(43) Pub. Date: Jan. 24, 2008

(54) N-[2-[[(DIAMINOMETHYLENE)AMINO]OXY]ETHYL]-3-[(2,2-DIFLUORO-2-PHENYLETHYL)AMINO]-6-METHYL-2-OXO-1(2H)-PYRAZINEACETAMIDE

(76) Inventors: Mitul N. Patel, Union, NJ (US); Christopher N. Nilsen, Branchburg, NJ (US); Mayra B. Reyes, Somerset, NJ (US); Michael Humora, Cranbury, NJ (US); Yun Qian, Doylestown, PA (US); Fuqiang Liu, Lansdale, PA (US); Xini Zhang, Bridgewater, NJ (US); Kirk Sorgi, Doylestown, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/751,155

(22) Filed: May 21, 2007

Related U.S. Application Data
(60) Provisional application No. 60/802,355, filed on May 22, 2006.
(60) Provisional application No. 60/835,320, filed on Aug. 3, 2006.

(51) Int. Cl. A61K 31/4965; A61P 7/02; C07D 241/08

(52) U.S. Cl. 514/255.06; 544/408

(57) ABSTRACT

The present invention is directed to N-[2-[[(diaminomethylene)amino]oxy]ethyl]-3-[(2,2-difluoro-2-phenylethyl)amino]-6-methyl-s-oxo-1(2H)-pyrazineacetamide, pharmaceutical compositions containing said compound and methods of treatment comprising inhibiting a serine protease using said compound. The present invention is further directed to a process for the preparation of the N-[2-[[(diaminomethylene)amino]oxy]ethyl]-3-[(2,2-difluoro-2-phenylethyl)amino]-6-methyl-2-oxo-1(2H)-pyrazineacetamide as a free base. The present invention is further directed to a process for the preparation of pharmaceutically acceptable salts of N-[2-[[(aminoiminomethyl)amino]oxy]ethyl]-3-[(2,2-difluoro-2-phenylethyl)amino]-6-methyl-2-oxo-1(2H)-pyrazineacetamide.

### CROSS REFERENCE TO RELATED APPLICATIONS

This application claims the benefit of U.S. Provisional Application Ser. No. 60/802,355, filed on May 22, 2006 and U.S. Provisional Application Ser. No. 60/835,320, filed on Aug. 03, 2006, which are incorporated by reference herein in their entirety.

### FIELD OF THE INVENTION

The present invention is directed to N-[2-[[(diaminomethylene)amino]oxy]ethyl]-3-[(2,2-difluoro-2-phenylethyl)amino]-6-methyl-2-oxo-1(2H)-pyrazineacetamide, pharmaceutical compositions containing said compound and methods of treatment comprising in particular the treatment of thrombosis using said compound.

The present invention is further directed to a process for the preparation of the N-[2-[[(diaminomethylene)amino]oxy]ethyl]-3-[(2,2-difluoro-2-phenylethyl)amino]-6-methyl-2-oxo-1(2H)-pyrazineacetamide as a base. The present invention is further directed to a process for the preparation of pharmaceutically acceptable salts of N-[2-[[(aminoiminomethyl)amino]oxy]ethyl]-3-[(2,2-difluoro-2-phenylethyl)amino]-6-methyl-2-oxo-1(2H)-pyrazineacetamide.

### BACKGROUND OF THE INVENTION

Lu et al., in U.S. Pat. No. 6,204,263 B1 disclose pyrazinone protease Inhibitors. The compounds disclosed in U.S. Pat. No. 6,204,263 B1 are potent inhibitors of proteases, especially trypsin-like serine proteases, such as chymotrypsin, trypsin, thrombin plasmin and factor Xa. Certain of the compounds exhibit antithrombic activity via direct, selective inhibition of thrombin. The compounds are further disclosed to be useful in methods of inhibiting treating aberrant proteolysis; and methods of treating thrombosis, ischemia, stroke, restenosis and inflammation. The compounds are further disclosed in pharmaceutical compositions for inhibiting loss of blood platelets, inhibiting formation of blood platelet aggregates, inhibiting formation of fibrin, inhibiting thrombus formation, and inhibiting embolus formation.

The compounds are further disclosed as useful in methods of inhibiting or treating aberrant proteolysis; treating myocardial infarction; unstable angina; stroke; restenosis; deep vein thrombosis; disseminated intravascular coagulation caused by trauma; sepsis or tumor metastasis; hemodialysis; cardiopulmonary bypass surgery; adult respiratory distress syndrome; endotoxic shock; rheumatoid arthritis; ulcerative colitis; induration; metastasis; hypercoagulability during chemotherapy; Alzheimer's disease; Down's syndrome; fibrin formation in the eye and wound healing. Other uses for the compound are as anticoagulants in the manufacture of devices used in blood collection, blood circulation, and blood storage such as catheters, blood dialysis machines, blood collection syringes and tubes, blood lines and stents.

The processes of the present invention are more efficient processes for the preparation of N-[2-[[(diaminomethylene)amino]oxy]ethyl]-3-[(2,2-difluoro-2-phenylethyl)amino]-6-methyl-2-oxo-1(2H)-pyrazineacetamide as a free base and pharmaceutically acceptable salts of N-[2-[[(aminoiminomethyl)amino]oxy]ethyl]-3-[(2,2-difluoro-2-phenylethyl)amino]-6-methyl-2-oxo-1(2H)-pyrazineacetamide. More particularly, some processes of the present invention allow for the preparation of said compounds with shorter reaction times, without isolation of non-solid, oily or foam intermediates, without chromatographic purification and/or in high yield and purity.

### SUMMARY OF THE INVENTION

The present invention is directed to a compound of formula (I)

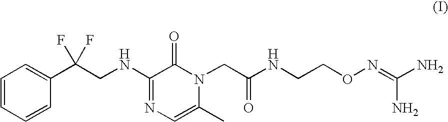

also known as N-[2-[[(diaminomethylene)amino]oxy]ethyl]-3-[(2,2-difluoro-2-phenylethyl)amino]-6-methyl-2-oxo-1(2H)-pyrazineacetamide; and pharmaceutically acceptable solvates, hydrates and pro-drugs thereof.

The present invention is further directed to process(es) for the preparation of the compound of formula (I), preferably wherein the compound of formula (I) is prepared as a base, more preferably as a substantially pure base, more preferably still, a base substantially free of any corresponding salt form.

In an embodiment, the present invention is directed to a process for the preparation of the compound of formula (I) comprising

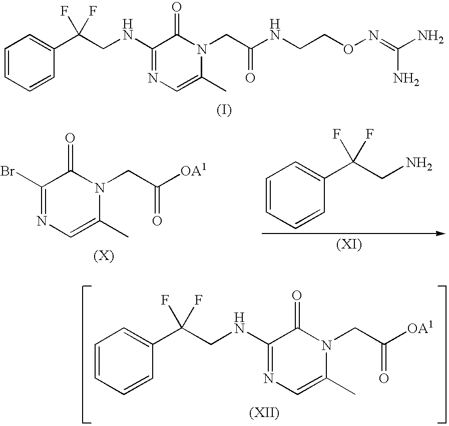

reacting a compound of formula (X), wherein A¹ is a lower alkyl, with a compound of formula (XI) or its corresponding HCl salt, in an organic solvent, to yield the corresponding compound of formula (XII);

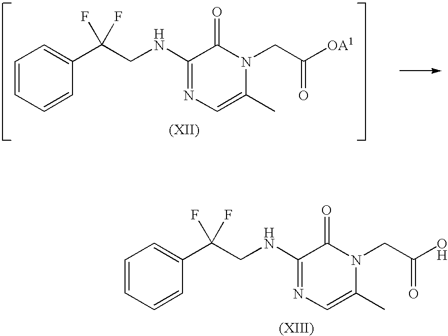

saponifying the compound of formula (XII) with an aqueous base, to yield the corresponding compound of formula (XIII);

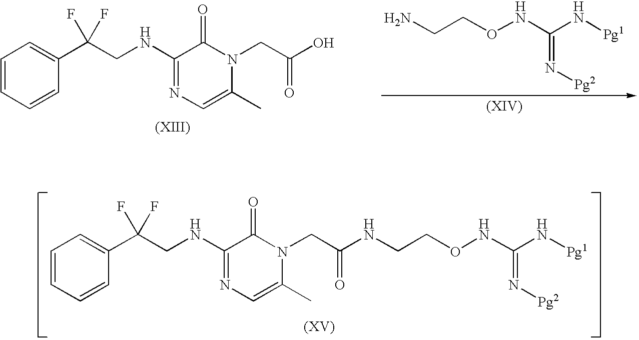

reacting the compound of formula (XIII) with a compound of formula (XIV) or its corresponding HCl salt, wherein Pg¹ and pg² are nitrogen protecting groups, in the presence of a coupling agent, in an organic solvent, to yield the corresponding compound of formula (XV);

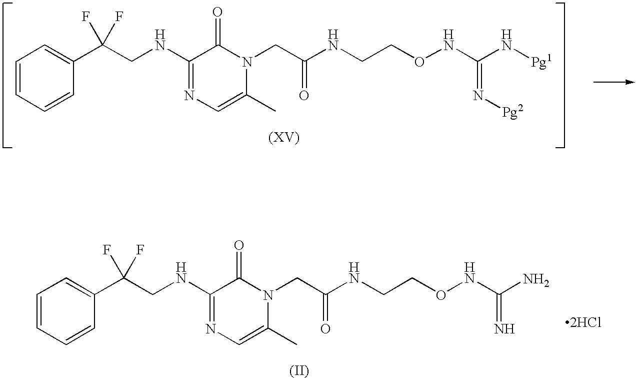

reacting the compound of formula (XV) with HCl, to yield the corresponding di-hydrochloride salt of the compound of formula (II);

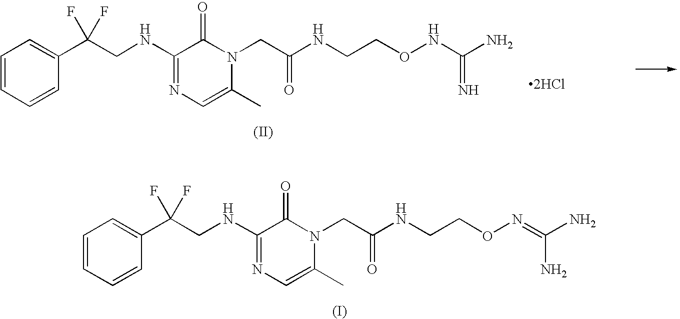

reacting the di-hydrochloride salt of the compound of formula (II) with a base, in water or a mixture of solvents, to yield the corresponding compound of formula (I).

The present invention is further directed to at least two crystalline forms of the compound of formula (I). Preferably, the crystalline forms of the compound of formula (I) are suitable for formulation in a pharmaceutical composition. The present invention is further directed to an anhydrous, non-hygroscopic crystalline form of the compound of formula (I), which form may be isolated as plates, needles, or a mixture thereof, preferably, as plates or needles, more preferably as needles. The present invention is further directed to a crystalline dichloromethane solvate form of the compound of formula (I).

The present invention is further directed to process(es) for the preparation and/or purification of the crystalline forms of the compound of formula (I).

The present invention is further directed to process(es) for the preparation of salts of the compound of formula (II)

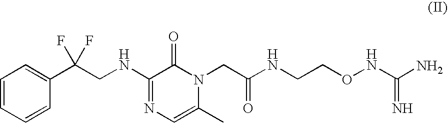

also known as N-[2-[[(aminoiminomethyl)amino]oxy]ethyl]-3-[(2,2-difluoro-2-phenylethyl)amino]-6-methyl-2-oxo-1(2H)-pyrazineacetamide.

The present invention is further directed to salts of the compound of formula (II), which may be amorphous, partially crystalline or crystalline. Preferably the salt of the compound of formula (II) is a di-HCl, di-HBr, di-TFA, tosylate, mesylate, nitrate, ethane-sulfonate or sulfate salts. Additionally, the salt of the compound of formula (II) may be a malonate or citrate co-crystal. Preferably, the salt of the compound of formula (II) is a di- or bis-salt. The present invention is further directed to solvates and hydrates of the salts and/or co-crystals of the compound of formula (II).

The present invention is further directed to at least two crystalline forms of the di-hydrochloride salt of the compound of formula (II). Preferably, the crystalline forms of the compound of formula (II) are suitable for formulation in a pharmaceutical composition. In an embodiment, the present invention is directed to a crystalline, di-hydrate, di-hydrochloride salt of the compound of formula (II). In another embodiment, the present invention is directed to a crystalline, anhydrous, di-hydrochloride salt of the compound of formula (II).

The present invention is further directed to process(es) for the preparation and/or purification of the crystalline forms of the di-hydrochloride salt of the compound of formula (II).

Illustrative of the invention is a pharmaceutical composition comprising a pharmaceutically acceptable carrier and the compound of formula (I). An illustration of the invention is a pharmaceutical composition made by mixing the compound of formula (I) and a pharmaceutically acceptable carrier. Illustrating the invention is a process for making a pharmaceutical composition comprising mixing the compound of formula (I) and a pharmaceutically acceptable carrier.

Exemplifying the invention are methods of inhibiting a serine protease comprising administering to a subject in need thereof a therapeutically effective amount of the compound of formula (I). Further exemplifying the invention are methods of inhibiting a serine protease selected from the group consisting of leukocyte neutrophil clastase, chymotrypsin, trypsin, pancreatic clastase, cathepsin G, thrombin, factor Xa, thermolysin and pepsin (preferably thrombin, more preferably human α-thrombin) comprising administering to a subject in need thereof a therapeutically effective amount of the compound of formula (I).

Further exemplifying the invention is a method of treating aberrant proteolysis due to a serine protease comprising administering to a subject in need thereof a therapeutically effective amount of the compound of formula (I). Further exemplifying the invention is a method of reducing blood coagulation comprising administering to a subject in need thereof a therapeutically effective amount of the compound of formula (I).

Exemplifying the invention is a method of treating thrombosis (preferably arterial and/or venous thrombosis) comprising administering to a subject in need thereof a therapeutically effective amount of the compound of formula (I). Further exemplifying the invention is a method of treating thrombosis, wherein the thrombosis is associated with or a result of one or more of ischemia, viral infection, stroke, restenosis, myocardial infarction, complication associated with artificial heart valves, complication associated with a stent, arterial fibrillation, uneven blood flow or pooling, disseminated intramuscular coagulopathy which occurs during septic shock, unstable angina, disseminated intramuscular coagulation caused by trauma, coronary artery bypass, hip replacement, thrombolytic therapy, sepsis, hemodialysis, adult respiratory distress syndrome, rheumatoid arthritis, ulcerative colitis, induration, metastasis, hypercoagulability during chemotherapy, fibrin formation in the eye and wound healing (preferably thrombosis associated with ischemia, restenosis, myocardial infarction, coronary artery bypass, hip replacement, thrombolytic therapy or wound healing) comprising administering to a subject in need thereof a therapeutically effective amount of the compound of formula (I).

Illustrative of the invention is a pharmaceutical composition comprising a pharmaceutically acceptable carrier and a salt of the compound of formula (II). An illustration of the invention is a pharmaceutical composition made by mixing a salt of the compound of formula (II) and a pharmaceutically acceptable carrier. Illustrating the invention is a process for making a pharmaceutical composition comprising mixing a salt of the compound of formula (II) and a pharmaceutically acceptable carrier.

Exemplifying the invention are methods of inhibiting a serine protease comprising administering to a subject in need thereof a therapeutically effective amount of a salt of the compound of formula (II). Further exemplifying the invention are methods of inhibiting a serine protease selected from the group consisting of leukocyte neutrophil clastase, chymotrypsin, trypsin, pancreatic clastase, cathepsin G, thrombin, factor Xa, thermolysin and pepsin (preferably thrombin, more preferably human α-thrombin) comprising administering to a subject in need thereof a therapeutically effective amount of a salt of the compound of formula (II).

Further exemplifying the invention is a method of treating aberrant proteolysis due to a serine protease comprising administering to a subject in need thereof a therapeutically effective amount of a salt of the compound of formula (II). Further exemplifying the invention is a method of reducing blood coagulation comprising administering to a subject in need thereof a therapeutically effective amount of a salt of the compound of formula (II).

Exemplifying the invention is a method of treating thrombosis (preferably arterial and/or venous thrombosis) comprising administering to a subject in need thereof a therapeutically effective amount of a salt of the compound of formula (II). Further exemplifying the invention is a method of treating thrombosis, wherein the thrombosis is associated with or a result of one or more of ischemia, viral infection, stroke, restenosis, myocardial infarction, complication associated with artificial heart valves, complication associated with a stent, arterial fibrillation, uneven blood flow or pooling, disseminated intramuscular coagulopathy which occurs during septic shock, unstable angina, disseminated intramuscular coagulation caused by trauma, coronary artery bypass, hip replacement, thrombolytic therapy, sepsis, hemodialysis, adult respiratory distress syndrome, rheumatoid arthritis, ulcerative colitis, induration, metastasis, hypercoagulability during chemotherapy, fibrin formation in the eye and wound healing (preferably thrombosis associated with ischemia, restenosis, myocardial infarction, coronary artery bypass, hip replacement, thrombolytic therapy or wound healing) comprising administering to a subject in need thereof a therapeutically effective amount of a salt of the compound of formula (II).

The present invention is further directed to a product prepared according to the process described herein.

Illustrative of the invention is a pharmaceutical composition comprising a pharmaceutically acceptable carrier and the product prepared according to any of the processes described herein. An illustration of the invention is a pharmaceutical composition made by mixing the product prepared according to any of the processes described herein and a pharmaceutically acceptable carrier. Illustrating the invention is a process for making a pharmaceutical composition comprising mixing the product prepared according to any of the processes described herein and a pharmaceutically acceptable carrier.

Another example of the invention is the use of any of the compounds described herein as a medicine. Yet another example of the invention is the use of any of the compounds described herein in the preparation of a medicament for treating any of the disorders as herein described, in a subject in need thereof.

### BRIEF DESCRIPTION OF THE FIGURES

FIG. 1 illustrates a representative XRD pattern for the anhydrous crystalline form of the compound of formula (I) (Form I-A).

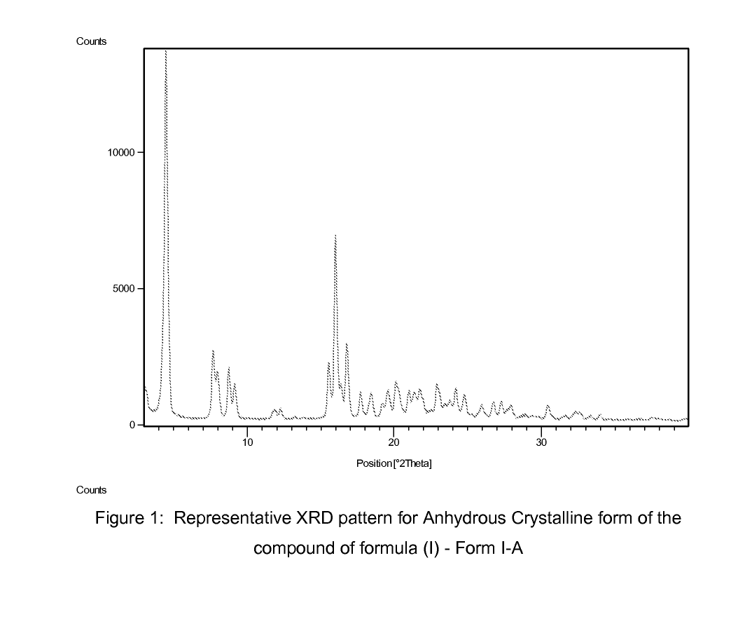

FIG. 2 illustrates a representative DSC trace for the anhydrous crystalline form of the compound of formula (I) (Form I-A).

FIG. 3 illustrates a representative TGA trace for the anhydrous crystalline form of the compound of formula (I) (Form I-A).

FIG. 4 illustrates a representative XRD pattern for the DCM solvate crystalline form of the compound of formula (I) (Form I-B).

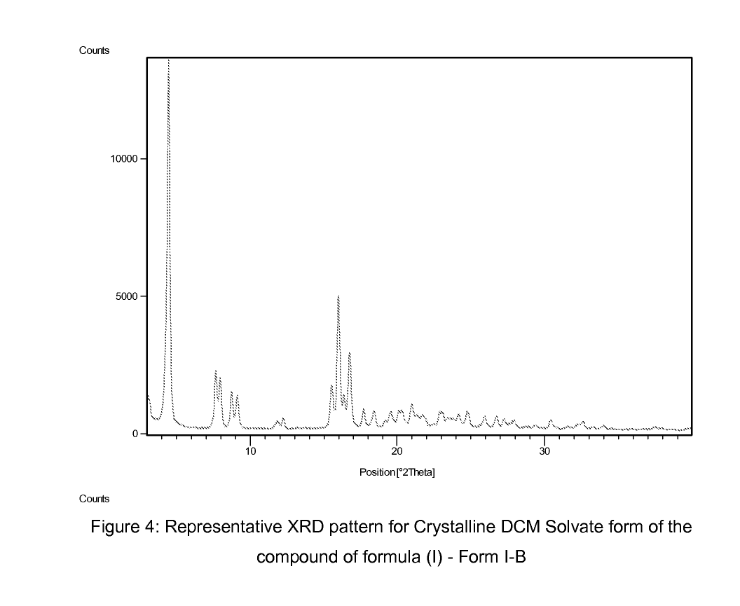

FIG. 5 illustrates a representative DSC pattern for the DCM solvate crystalline form of the compound of formula (I) (Form I-B).

FIG. 6 illustrates a representative TGA pattern for the DCM solvate crystalline form of the compound of formula (I) (Form I-B).

FIG. 7 illustrates a representative XRD pattern for the crystalline di-hydrate form of the di-hydrochloride salt of the compound of formula (II) (Form II-A).

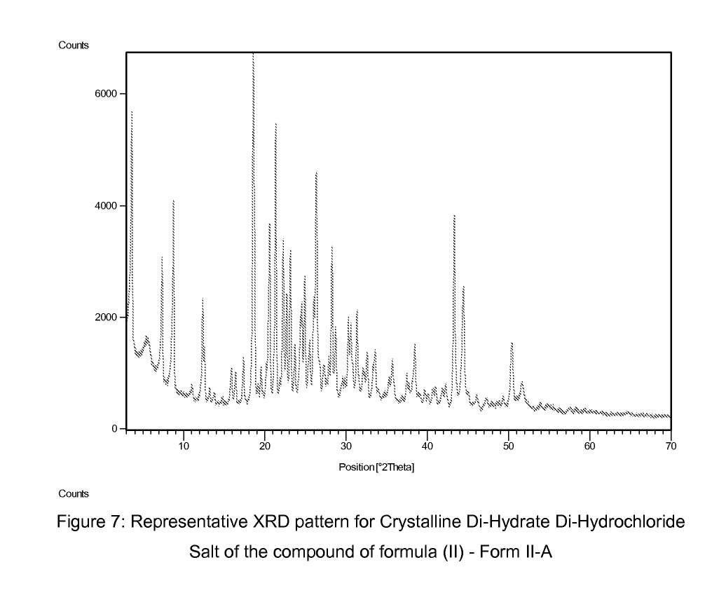

FIG. 8 illustrates a representative TGA trace for the crystalline di-hydrate form of the di-hydrochloride salt of the compound of formula (II) (Form II-A).

FIG. 9 illustrates a representative XRD pattern for the crystalline anhydrous form of the di-hydrochloride salt of the compound of formula (II) (Form II-B).

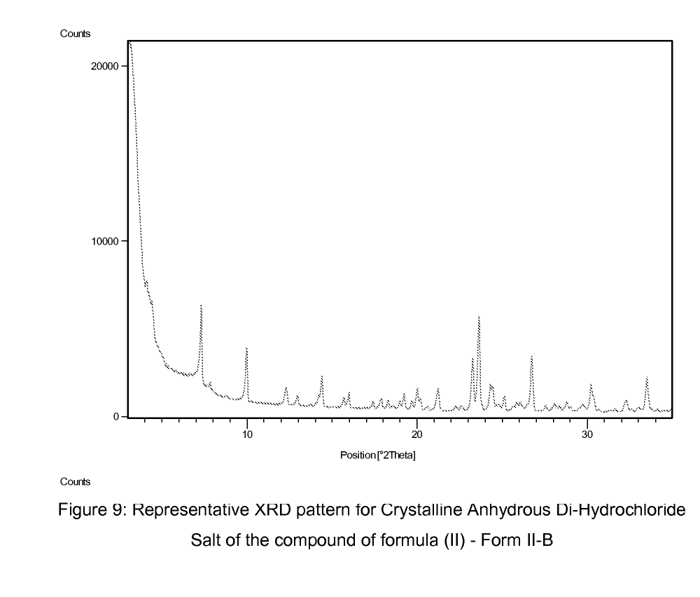

FIG. 10 illustrates a representative TGA trace for the crystalline anhydrous form of the di-hydrochloride salt of the compound of formula (II) (Form II-B).

### DETAILED DESCRIPTION OF THE INVENTION

The present invention is directed the compound of formula (I)

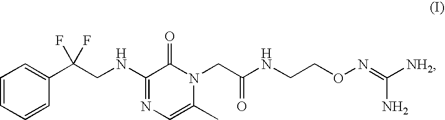

also known as N-[2-[[(diaminomethylene)amino]oxy]ethyl]-3-[(2,2-difluoro-2-phenylethyl)amino]-6-methyl-2-oxo-1(2H)-pyrazineacetamide. The compound of formula (I) is a tautomer of the compound of formula (II)

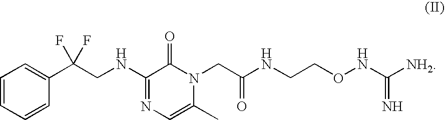

A compound of formula (II) as a TFA salt is disclosed in U.S. Pat. No. 6,204,263 B1 (Issued Mar. 20, 2001). The compound of formula (II) is further disclosed as a potent inhibitor of proteases, especially trypsin-like serine proteases such as chymotrypsin, trypsin, thrombin, plasmin and factor Xa.

In an embodiment of the present invention, the compound of formula (I) is substantially pure. In another embodiment of the present invention, the compound of formula (I) is substantially free of any of its corresponding salt form(s). In another embodiment of the present invention, the compound of formula (I) is present in an isolated form.

In an embodiment of the present invention, the salt of the compound of formula (II) is substantially pure. In another embodiment of the present invention, the salt of the compound of formula (II) is substantially free of any other salt form(s). In another embodiment of the present invention, the salt of the compound of formula (II) is present in an isolated form.

In an embodiment of the present invention, the salts of the compound of formula (II) are pharmaceutically acceptable, in particular when salt of the compound of formula (II) is used as the active ingredient in the medicament. In another embodiment the salts of the compound of formula (II) are used as intermediates in synthesis processes. In that case said salts of the compound of formula (II) do not necessarily have to be pharmaceutically acceptable. In another embodiment, the present invention is directed to pharmaceutical compositions comprising at least one of the pharmaceutically acceptable salts of the compound of formula (II) and a pharmaceutically acceptable carrier. In another embodiment, the present invention is directed to a pharmaceutical composition made by mixing at least one of the pharmaceutically acceptable salts of the compound of formula (II) and a pharmaceutically acceptable carrier. Illustrating the invention is a process for making a pharmaceutical composition comprising mixing at least one of the pharmaceutically acceptable salts of the compound of formula (II) and a pharmaceutically acceptable carrier.

The present invention is further directed to process(es) for the preparation of the compound of formula (I). The present invention is further directed to a process for the preparation of pharmaceutically acceptable salts of the compound of formula (II).

In an embodiment, the present invention is directed to a process for the preparation of a di-hydrochloride, di-hydrobromide, di-TFA, tosylate, mesylate, nitrate, ethane-sulfonate or sulfate salt of the compound of formula (II), preferably a di-hydrochloride salt of the compound of formula (II), more preferably a crystalline, di-hydrate, di-hydrochloride salt of the compound of formula (II). In another embodiment, the present invention is directed to a process for the preparation of a malonate or citrate co-crystal salts of the compound of formula (II).

The present invention is further directed to at least one, preferably at least two, novel crystalline forms of the compound of formula (I). Preferably, the crystalline form of the compound of formula (I) is anhydrous. Preferably, the crystalline form of the compound of formula (I) is non-hygroscopic. The present invention is further directed to an anhydrous crystalline form of the compound of formula (I) which is needle-like in appearance. The present invention is further directed to an anhydrous crystalline form of the compound of formula (I) which is plate-like in appearance. The present invention is further directed to a crystalline dichloromethane solvate form of the compound of formula (I).

The present invention is further directed to at least one, preferably at least two, crystalline forms of the di-hydrochloride salt of the compound of formula (II). In an embodiment of the present invention, the di-hydrochloride salt of the compound of formula (II) is a di-hydrate. In another embodiment of the present invention, the di-hydrochloride salt of the compound of formula (II) is anhydrous.

The present invention is further directed to co-therapy comprising administration to a subject in need thereof a therapeutically effective amount of the compound of formula (I) and one or more anti-thrombosis (anti-thrombin agent) agents. Suitable examples of anti-thrombosis agents include factor Xa agents, anti-platelet agents (including, but not limited to clopidegrel, aspirin, and the like), and the like.

Definitions

A tautomer is a structural isomer that is the result of a process called tautomerism.

Tautomerism is the reversible inter-conversion of structural isomers, usually involving the transfer of a proton. A comparison of the chemical structure of tautomers shows that their atoms are arranged differently. In theory, by selecting specific isolation and/or purification conditions, the individual and/or mixture of tautomers may be stabilized and isolated. Further, in solution, depending on the solvent system, a compound that can tautomerize may be present in one or more of its tautomeric forms.

For compounds which comprise a guanidine substituent group, a group of the following structure, denoted (G-1)

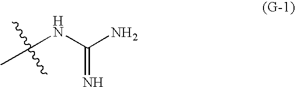

a proton (hydrogen) of the NH₂ group may be expected to migrate to the ═NH portion of the guanidine group, as shown in the schematic below, to form the following tautomeric structure denoted (G-T1)

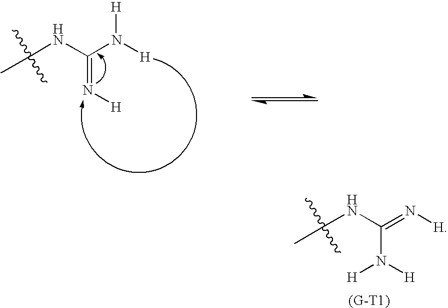

In the compound of formula (I) of the present invention, the guanidine group (G-1) was determined to undergo the tautomerization as shown in the schematic below, to yield the tautomeric form of the guanidine group of following structure, denoted (G-T2)

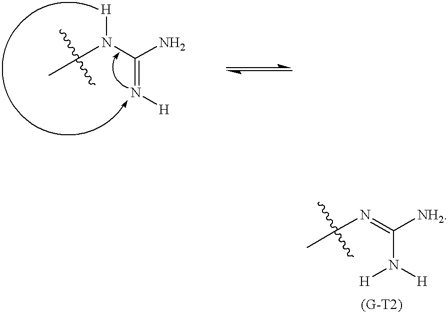

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

As used herein, unless otherwise noted, the term “nitrogen protecting group” shall mean a group which may be attached to a nitrogen atom to protect said nitrogen atom from participating in a reaction and which may be readily removed following the reaction. Suitable nitrogen protecting groups include, but are not limited to carbamates—groups of the formula —C(O)O—R wherein R is for example methyl, ethyl, t-butyl, benzyl, phenylethyl, CH₂═CH—CH₂—, and the like; amides—groups of the formula —C(O)—R′ wherein R′ is for example methyl, phenyl, trifluoromethyl, and the like; N-sulfonyl derivatives—groups of the formula —SO₂—R″ wherein R″ is for example tolyl, phenyl, trifluoromethyl, 2,2,5,7,8-pentamethylchroman-6-yl-, 2,3,6-trimethyl-4-methoxybenzene, and the like. Other suitable nitrogen protecting groups may be found in texts such as T. W. Greene & P. G. M. Wuts, Protective Groups in Organic Synthesis, John Wiley & Sons, 1991.

Abbreviations used in the specification, particularly the Schemes and Examples, are as follows:

| | |
|---|---|
| BOC or Boc = | t-butoxycarbonyl |
| BOP = | (Benzotriazol-1-yloxy)tris- |
| | (dimethylamino)phosphonium |
| | hexafluorophosphate |
| Cbz = | Benzyloxycarbonyl |
| CDMT | 22-chloro-4,6-dimethoxy-1,3,5-triazine |
| DBU = | Diaza(1,3)bicyclo[5.4.0]undecane |
| DCC = | Dicyclohexylcarbodiimide |
| DCM = | Dichloromethane |
| DMAP = | Dimethylaminopyridine |
| DSC = | Differential Scanning Calorimetry |
| EDCl = | 1-Ethyl-3-(3′- |
| | dimethylaminopropyl)carbodiimide |
| | hydrochloride |
| HOBt = | 1-Hydroxybenzotriazole |
| IBCF = | Isobutylchloroformate |
| ⁱPr₂Net = | Diisopropylethylamine |
| LCMS = | Liquid Chromatography Mass Spectrometry |
| MP = | Melting point |
| NMM = | N-Methylmorpholine |
| NMP = | N-methyl-2-pyrrolidinone |
| NMR = | Nuclear Magnetic Resonance |
| TEA = | Triethylamine |
| TFA = | Trifluoroacetic Acid |
| TGA = | Thermogravimetric Analysis |
| THF = | Tetrahydrofuran |
| XRD = | X-Ray Diffraction |

Serine proteases include, but are not limited to neutrophil clastase, chymotrypsin, trypsin, pancreaatic clastase, cathepsin G, thrombin, factor Xa, thermolysin and pepsin. Preferably, the serine protease is thrombin.

As used herein, unless otherwise noted, the term “thrombosis” shall mean the formation of a blood clot, regardless of associated or underlying cause and/or the place within the body where blood clot forms. In an embodiment, the blood clot comprises fibrin deposits, with or without platelets. In another embodiment, the thrombosis results in a significant clinical event such as pain, ischemia, stroke, myocardial infarction, pulmonary embolism, and the like.

For example, the thrombosis may be associated with or a result of ischemia, viral infection, stroke, restenosis, myocardial infarction, complication associated with artificial heart valves, complication associated with a stent, arterial fibrillation, uneven blood flow or pooling, disseminated intramuscular coagulopathy which occurs during septic shock, unstable angina, disseminated intramuscular coagulation caused by trauma, coronary artery bypass, hip replacement, thrombolytic therapy, sepsis, hemodialysis, adult respiratory distress syndrome, rheumatoid arthritis, ulcerative colitis, induration, metastasis, hypercoagulability during chemotherapy, fibrin formation in the eye and wound healing. Preferably, the thrombosis is associated with thrombosis associated with ischemia, restenosis, myocardial infarction, coronary artery bypass, hip replacement, thrombolytic therapy or wound healing. More preferably, the thrombosis is arterial and/or venous.

The term “subject” as used herein, refers to an animal, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of at least one of the symptoms of the disease or disorder being treated and/or reduction in the frequency and/or severity of at least one of the symptoms of the disease or disorder being treated.

Wherein the present invention is directed to co-therapy or combination therapy, comprising administration of (a) the compound of formula (I) or one or more of the salts of the compound of formula (II) or any mixture thereof and (b) one or more anti-thrombosis agents, therapeutically effective amount shall mean that amount of the combination of agents taken together so that the combined effect elicits the desired biological or medicinal response. For example, the therapeutically effective amount of co-therapy comprising administration of the compound of formula (I) and at least one suitable anti-thrombosis agent would be the amount of the compound of formula (I) and the amount of the suitable anti-thrombosis agent that when taken together or sequentially have a combined effect that is therapeutically effective. Further, it will be recognized by one skilled in the art that in the case of co-therapy with a therapeutically effective amount, as in the example above, the amount of the compound of formula (I) and/or the amount of the suitable anti-thrombosis agent individually may or may not be therapeutically effective.

As used herein, the term “composition” is intended to encompass a product comprising the specified ingredients in the specified amounts, as well as any product which results, directly or indirectly, from combinations of the specified ingredients in the specified amounts.

As used herein, unless otherwise noted, the term “substantially pure base” shall mean that the mole percent of impurities in the isolated base is less than about 5 mole percent, preferably less than about 2 mole percent, more preferably, less than about 0.5 mole percent, most preferably, less than about 0.1 mole percent.

As used herein, unless otherwise noted, the term “substantially free of a corresponding salt form(s)” when used to described the compound of formula (I) shall mean that mole percent of the corresponding salt form(s) in the isolated base of formula (I) is less than about 5 mole percent, preferably less than about 2 mole percent, more preferably, less than about 0.5 mole percent, most preferably less than about 0.1 mole percent.

As used herein, unless otherwise noted, the term “isolated form” shall mean that the compound is present in a form which is separate from any solid mixture with another compound(s), solvent system or biological environment.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Preferably, wherein the compound is present as an enantiomer, the enantiomer is present at an enantiomeric excess of greater than or equal to about 80%, more preferably, at an enantiomeric excess of greater than or equal to about 90%, more preferably still, at an enantiomeric excess of greater than or equal to about 95%, more preferably still, at an enantiomeric excess of greater than or equal to about 98%, most preferably, at an enantiomeric excess of greater than or equal to about 99%. Similarly, wherein the compound is present as a diastereomer, the diastereomer is present at an diastereomeric excess of greater than or equal to about 80%, more preferably, at an diastereomeric excess of greater than or equal to about 90%, more preferably still, at an diastereomeric excess of greater than or equal to about 95%, more preferably still, at an diastereomeric excess of greater than or equal to about 98%, most preferably, at an diastereomeric excess of greater than or equal to about 99%.

Furthermore, some of the crystalline forms for the compounds of the present invention may exist as polymorphs and as such are intended to be included in the present invention. In addition, some of the compounds of the present invention may form solvates with water (i.e., hydrates) or common organic solvents, and such solvates are also intended to be encompassed within the scope of this invention.

The present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

Processes for Preparation

The present invention is directed to processes for the preparation of a salt of the compound of formula (II) and the compound of formula (I), as described in more detail in Scheme 1 below.

One skilled in the art will recognize that wherein a reaction step of the present invention may be carried out in a variety of solvents or solvent systems, said reaction step may also be carried out in a mixture of the suitable solvents or solvent systems.

Where the processes for the preparation of the compounds according to the invention described hereunder give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

During any of the processes for preparation of the compounds of the present invention described hereunder, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

More specifically, as an example of the preparation of a salt of the compound of formula (II), Scheme 1 below outlines the preparation of the di-hydrochloride salt of the compound of formula (II) and further conversion of the di-hydrochloride salt of the compound of formula (II) to the compound of formula (I).

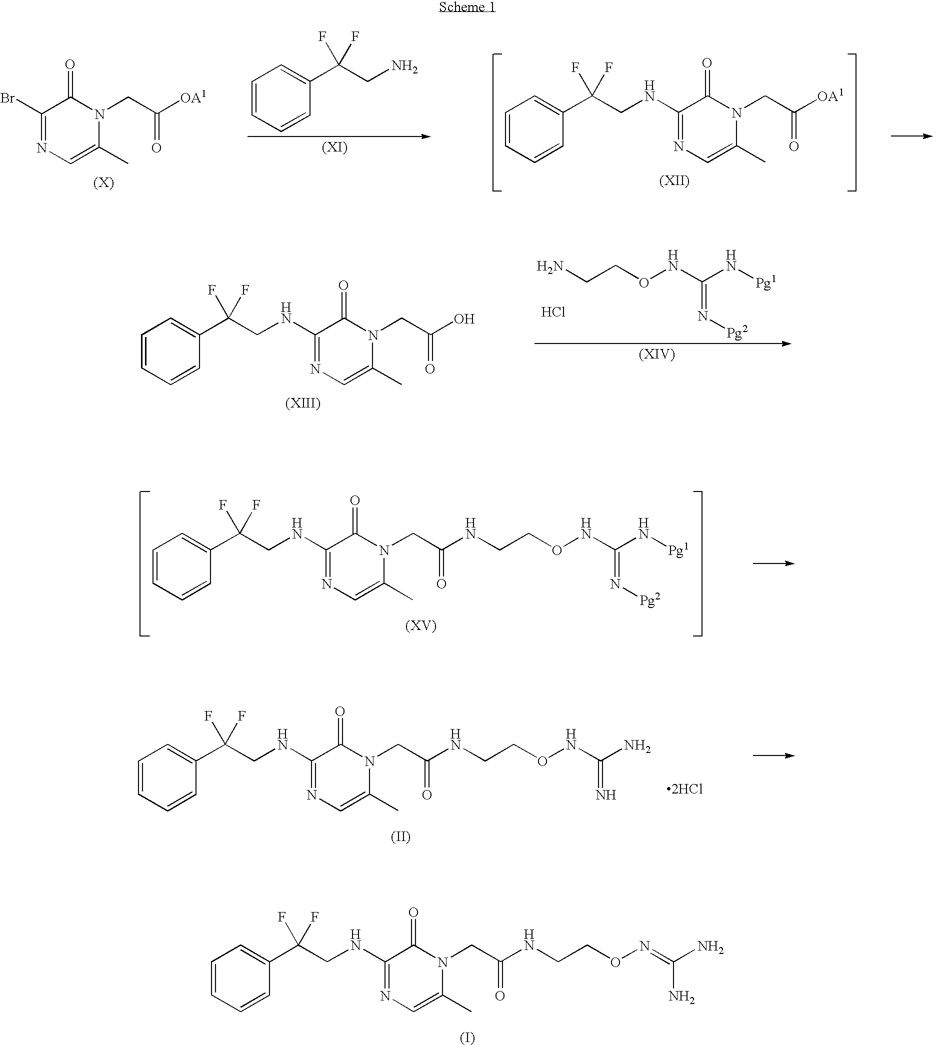

Accordingly, a suitably substituted compound of formula (X), wherein A¹ is a lower alkyl such as methyl, ethyl, propyl, isopropyl, butyl, isobutyl, t-butyl, and the like, preferably ethyl, a known compound or compound prepared by known methods, is reacted with the compound of formula (XI), also known as 2,2-difluoro-2-phenyl-ethylamine, a known compound, wherein the compound of formula (XI) is preferably, present in an amount greater than about 1 molar equivalent (relative to the moles of the compound of formula (X)), preferably in an amount in the range of from about 1.2 to about 3 molar equivalents, more preferably in an amount in the range of from about 1.2 to about 2.2 molar equivalents, most preferably, about 1.2 molar equivalents in production scale; preferably in the presence of an inorganic base, preferably in the presence of an inorganic base which does not liberate water, such as NaHPO₄, and the like, preferably NaHPO₄; wherein the inorganic base is preferably present in an amount greater than about 1.2 molar equivalents (relative to the moles of the compound of formula (X)), more preferably, about 1.2 molar equivalents; in a organic solvent such as ethanol, butanol, toluene, acetonitrile, NMP, and the like, preferably butanol; at a temperature greater than about room temperature, preferably at about (solvent) reflux temperature; to yield the corresponding compound of formula (XII).

Preferably, the compound of formula (XII) is not isolated. Optionally, the compound of formula (XII) is isolated and/or purified according to known methods.

The compound of formula (XII) is hydrolyzed according to known methods, for example by reacting with a base such as NaOH, KOH, LiOH, barium hydroxide, calcium hydroxide, t-butyl ammonium hydroxide, and the like, preferably an inorganic base such NaOH, KOH, and the like, more preferably, aqueous NaOH, more preferably, aqueous NaOH at a concentration of about 25% to about 30%; wherein the base is preferably present in an excess amount; in a polar organic solvent or mixture of polar organic solvent and water, solvent such as butanol, a mixture of ethanol and water, a mixture of THF and water, and the like, preferably butanol, more preferably in the same solvent as used in the previous reaction step (i.e. the step of reacting the compound of formula (X) with the compound of formula (Xi)); at a temperature in the range of about room temperature to about (solvent) reflux temperature, preferably when the solvent is butanol, the compound of formula (X) is reacted with the compound of formula (XI) at a temperature in the range of from about 75° C. to about 90° C., and when the solvent is a mixture of ethanol and water, the compound of formula (X) is reacted with the compound of formula (XI) at about room temperature; to yield the corresponding compound of formula (XIII).

Preferably, the compound of formula (XIII) is isolated according to known methods, including neutralizing with a suitable acid such as HCl, and the like, and then further isolating according to known methods, for example, filtration. Preferably, the filter cake is further washed, according to known methods, to remove soluble impurities. Alternatively, the compound of formula (XIII) is extracted according to known methods.

The compound of formula (XIII) is further optionally purified according to known methods, for example by washing with a solvent in which the compound of formula (XIII) is not highly soluble; by recrystallization; or other suitable method.

The compound of formula (XIII) is reacted with a suitably substituted compound of formula (XIV) or its corresponding salt, for example its corresponding HCl salt, wherein Pg¹ and pg² are suitably selected nitrogen protecting groups, such as Boc, Cbz, and the like, preferably Pg¹ and pg² are nitrogen protecting groups which may be removed by reacting with an acid, more preferably, Pg¹ and Pg² are the same nitrogen protecting group, more preferably, Pg¹ and pg² are each Boc, a known compound or compound prepared by known methods; wherein the compound of formula (XIV) is preferably, present in an amount equal to about 1 molar equivalent, more preferably, in an amount in the range of about 1.05 to about 2.0 molar equivalents, more preferably in an amount in the range of about 1.05 to about 1.25 molar equivalents; in the presence of a suitably selected coupling agent such as BOP, DCC/HOBt, CDMT (wherein the CDMT is preferred when the compound of formula (XIV) is its corresponding HCl salt) and the like, preferably CDMT or DCC/HOBt, more preferably mixture of DCC and about 5 mole % HOBt; wherein the pH of the reaction mixture is preferably maintained in the range of from about 5 to about 7, more preferably, at a pH in the range of from about 5.25 to about 6.5, more preferably, at pH in the range of from about 5.5 to about 6, using a suitably selected acid or base such as dilute aqueous HCl, dilute aqueous NaOH, NMM, and the like, preferably, NMM; in an organic solvent such as THF, DMF, acetonitrile, a mixture of acetonitrile and water, and the like, preferably in DMF, THF or acetonitrile, more preferably in a mixture of acetonitrile and water; preferably at about room temperature; to yield the corresponding compound of formula (XV).

Preferably, the compound of formula (XV) is not isolated. Optionally, the compound of formula (XV) is isolated and/or purified according to known methods.

One skilled in the art will recognize that the compound of formula (XV) may be isolated by precipitation. One skilled in the art will further recognize that isolation of the compound of formula (XV) may be preferred when the solvent in which the compound of formula (XV) is prepared is incompatible with the conditions of de-protection used in the next step. For example, wherein the compound of formula (XV) is prepared in a solvent such as acetonitrile and wherein the de-protection is under acidic conditions.

One skilled in the art will recognize that the compound of formula (XIII) may alternatively be reacted with the unprotected analog of the compound of formula (XIV), a compound of formula (XIV-UP)

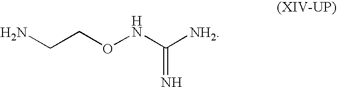

This reaction would however, be expected to yield a mixture of products (resulting from the coupling of the acid portion of the compound of formula (XIII) with one or more of the three terminal nitrogens on the unprotected analog of the compound of formula (XIV)), resulting in lower yields of the desired compound and/or difficult separation of the desired product from undesired by-products.

The compound of formula (XV) is de-protected according to known methods, for example, by reacting with an acid such as HCl(g), HCl(aq) (preferably, about 15 to about 30%), TFA, H₂SO₄, nitric acid, ethanesulfonic acid, phosphoric acid, HI, HBr, TsOH, and the like, preferably HCl (aq) or HCl(g), more preferably HCl(aq), more preferably about 20% HCl(aq), wherein the acid is preferably present in an excess amount; in an organic solvent such as THF, dioxane, water, and the like, preferably in THF or water, more preferably water; to yield the corresponding salt of the compound of formula (II).

In the above scheme, the compound of formula (XV) is de-protected according to known methods, by reacting, preferably, via reverse addition, with aqueous HCl, to yield the corresponding di-hydrochloride salt of the compound of formula (II).

One skilled in the art will recognize that wherein a salt other than the di-hydrochloride salt of the compound of formula (II) is desired, the compound of formula (XV) is reacted with desired acid. For example, wherein a TFA salt is desired, the compound of formula (XV) is de-protected by reacting with TFA, preferably an excess amount of TFA to yield the corresponding di-TFA salt of the compound of formula (II). Alternatively, a salt of the compound of formula (II) other than the di-hydrochloride salt may be prepared from the di-hydrochloride salt of the compound of formula (II) according to known methods, for example by ion exchange.

The salt of the compound of formula (II) is optionally isolated according to known methods, for example, by filtration. The salt of the compound of formula (II) is further, optionally, purified according to known methods, for example by washing with a solvent in which the salt of the compound of formula (II) is not highly soluble; by charcoal treatment; by recrystallization from a suitable solvent or mixture thereof such as methanol:ethyl acetate, methanol:water:ethyl acetate, IPA:water, IPA:ethyl acetate, and the like; or other suitable method.

In an embodiment of the present invention, the di-hydrochloride salt of the compound of formula (II) is re-crystallized from a mixture of methanol, water and ethyl acetate, to yield the corresponding di-hydrate form of the di-hydrochloride salt of the compound of formula (II). In an embodiment, the ratio of methanol: water: ethyl acetate is about 1.5: about 0.5: about 5.5. In another embodiment, the ratio of methanol:water:ethyl acetate is about 1.3:about 0.7:about 4.0.

In another embodiment of the present invention, the di-hydrochloride salt of the compound of formula (II) is re-crystallized from a mixture of methanol and ethyl acetate, to yields the anhydrous form of the di-hydrochloride salt of the compound of formula (II). In an embodiment, the ratio of methanol:ethyl acetate is about 2.0 about 3.0. In another embodiment, the ratio of methanol ethyl acetate is about 1.5:about 3.5. In another embodiment, the ratio of methanol ethyl acetate is about 1.0:about 3.4.

One skilled in the art will recognize that by selecting and varying the solvent type and/or mixture amounts, different polymorph, hydrate, solvate and co-crystal forms of the salt of the compound of formula (II) may be obtained.

Alternatively, the compound of formula (II), as its corresponding salt (for example its corresponding di-hydrochloride salt as outlined in the Scheme above) is not isolated.

The compound of formula (II), as its corresponding salt is reacted with a suitably selected base such as NaOH, LiOH, Ca(OH)₂, Ba(OH)₂, Na₂CO₃, tetrabutyl ammonium hydroxide, and the like, preferably an inorganic base such as NaOH, Na₂CO₃, and the like, more preferably, NaOH or Na₂CO₃, more preferably, Na₂CO₃; wherein the base is preferably present in an excess amount; in water or a mixture of solvents such as a mixture of methanol and water, and the like, preferably, in water; to yield the corresponding compound of formula (I), as a base, preferably, as the anhydrous crystalline form of the compound of formula (I).

Preferably, the compound of formula (I) is isolated according to known methods, for example by filtration. Preferably, the compound of formula (I) is purified according to known methods, for example by washing with a solvent in which the compound of formula (I) is not highly soluble; by charcoal treatment to remove colored impurities, by recrystallization from a suitable solvent or mixture of solvents such as methanol:water; and/or by any other suitable method.

In an embodiment, the compound of formula (I) is re-crystallized according to known methods, from a suitably selected solvent or mixture thereof such as a mixture of methanol and water, and the like. In an embodiment, the compound of formula (I) is dissolved in a mixture of about 80:20 of methanol:water, the resulting mixture is heated, filtered, additional water is added to a final mixture of about 33:67 methanol:water, and the mixture cooled, resulting in the precipitation of substantially pure compound of formula (I), wherein the compound of formula (I) is isolated as the anhydrous crystalline form of the compound of formula (I), as needles.

Crystalline Forms

The present invention is further directed to novel crystalline forms of the compound of formula (I). In an embodiment, the present invention is directed to at least one, preferably at least two crystalline forms of the compound of formula (I). In an embodiment of the present invention, the crystalline form of the compound of formula (I) is isolated as needles. In another embodiment of the present invention, the crystalline form of the compound of formula (I) is isolated as platelets. Preferably, the crystalline form of the compound of formula (I) is needles, which form is easier to filter. In an embodiment of the present invention, the crystalline form of the compound of formula (I) is anhydrous. In another embodiment of the present invention, the crystalline form of the compound of formula (I) is a DCM solvate.

The present invention is further directed to novel crystalline forms of the di-hydrochloride salt of the compound of formula (II). In an embodiment, the present invention is directed to at least one, preferably at least two crystalline forms of the di-hydrochloride salt of the compound of formula (II), hereinafter referred to as Form II-A and Form II-B. In an embodiment of the present invention, the crystalline form of the di-hydrochloride salt of the compound of formula (II) is a di-hydrate. In another embodiment of the present invention, the crystalline form of the di-hydrochloride salt of the compound of formula (II) is anhydrous.

The di-hydrochloride salts of the compound of formula (II), regardless of crystalline form are at least somewhat hygroscopic. The di-hydrate form of the di-hydrochloride salt of the compound of formula (II) exhibits a stable XRD pattern over the range of 6-8% water as measured by Karl-Fischer.

The crystalline forms of the present invention were characterized by their respective x-ray powder diffraction patterns utilizing a diffractometer using CuK_{α} radiation and suitable system conditions, for example as listed below:
a) CuKα radiation, 30 mA, 40 KV
b) 1/12° divergence slit, 0.2 receiving slit
c) Scanning from 4 to 35°2θ at a scan rate of 0.016°2θ/second
d) Aluminum sample holder

The crystalline forms of the compound of formula (I) and/or the salts of the compound of formula (II) may be characterized by their XRD spectra which may be identified by their 2θ, d-spacing and optionally their relative intensity values.

In an embodiment, the crystalline forms of the compound of formula (I) and/or the salts of the compound of formula (II) may be characterized by their respective XRD spectra comprising peaks with a relative intensity of greater than or equal to about 10%, preferably about 25%.

In an embodiment, the present invention is directed to an anhydrous, crystalline form of the compound of formula (I) isolated as needles, plates or a mixture thereof, hereinafter referred to as Form I-A.

The crystalline form of the compound of formula (I) isolated as needles may be prepared from the di-hydrate, di-hydrochloride salt of the compound of formula (II) by controlling the ratio of methanol:water: sodium hydroxide used in the conversion. For example, the crystalline form of the compound of formula (I) isolated as needles may be prepared by neutralizing a solution of the di-hydrochloride salt of the compound of formula (II) by treating the di-hydrochloride salt of the compound of formula (II) with an excess, preferably about 1 mole excess, of 1 N sodium hydroxide in a mixture of water and methanol at a methanol concentration of 80-90 mg/mL at a temperature of about 55-60° C. followed by a slow cool down of the resulting mixture to a temperature of about 20-22° C. The needles of the crystalline form of the compound of formula (I) are easily filtered (as compared with the plates of the crystalline form of the compound of formula (I)).

The crystalline form of the compound of formula (I) isolated as platelets may be prepared for example by dissolving the di-hydrate di-hydrochloride salt of the compound of formula (II) in water and neutralizing with an excess, preferably about 1 mole excess, of 1 N sodium hydroxide. When the pH of the resulting mixture reaches a pH in the range of from about 9 to about 14, the compound of formula (I) crystallizes out as plates, which plates are difficult to filter.

Both the needle and plate forms of the crystalline form of the compound of formula (I) were determined to be anhydrous as measured by TGA measurement. Further, the XRD patterns measured for both the needle and plate forms of the crystalline form of the compound of formula (I) indicated that although the two forms are different at a microscopic level, the crystal structure for the two forms (at an atomic level) are the same. In other words the needle and plate forms are two different crystal habits. Preferably, the anhydrous crystalline Form I-A of the compound of formula (I) is isolated and/or prepared in its needle form.

Crystalline Form I-A may be characterized by its XRD spectra which comprises the following peaks:

TABLE 1
Form I-A:
Anhydrous Crystalline Form of the Compound of Formula (I)

| Pos. [°2Th.] | d-spacing [Å] | Rel. Int. [%] |
|---|---|---|
| 4.46 | 19.8 | 100.0 |
| 7.64 | 11.6 | 16.6 |
| 7.97 | 11.1 | 12.8 |
| 8.72 | 10.1 | 12.9 |
| 9.16 | 9.7 | 9.5 |
| 15.53 | 5.7 | 15.6 |
| 15.97 | 5.6 | 49.6 |
| 16.73 | 5.3 | 20.7 |
| 20.11 | 4.4 | 10.4 |

The crystalline DCM solvate form of the compound of formula (I), hereinafter referred to as Form I-B, may be prepared from the corresponding needle or plate form of the compound of formula (I) by dissolving the compound of formula (I) in DCM, stirred or sonicated for about 1-2 minutes, and then all solvents were allowed to evaporate under ambient condition.

Crystalline Form I-B may be characterized by its XRD spectra which comprises the following peaks:

TABLE 2
Form I-B:
DCM Solvate Crsytalline Form of the Compound of Formula (I)

| Pos. [°2Th.] | d-spacing [Å] | Rel. Int. [%] |
|---|---|---|
| 4.47 | 19.8 | 100.0 |
| 7.65 | 11.6 | 1530 |
| 7.95 | 11.1 | 13.5 |
| 8.71 | 10.2 | 9.4 |
| 9.13 | 9.7 | 8.9 |
| 15.52 | 5.7 | 11.6 |
| 15.97 | 5.6 | 35.6 |
| 16.74 | 5.3 | 20.2 |

In an embodiment, the present invention is directed to two crystalline forms and one amorphous form of the di-hydrochloride salt of the compound of formula (II). More specifically, Form II-A is crystalline and di-hydrate (as measured by TGA); and Form II-B is crystalline and anhydrous.

Crystalline Form II-A may be prepared by re-crystallizing the di-hydrochloride salt, prepared according to any of the processes described herein from a mixture of IPA and water. In an embodiment, the ratio of IPA:water is about 8.0: about 1.5. Alternatively, crystalline Form II-A may be prepared from the anhydrous form of the di-hydrochloride salt of the compound of formula (II) by dissolving the salt in a mixture of methanol:water, as described above, and then adding ethyl acetate as an anti-solvent. Alternatively, crystalline Form II-A may be prepared from the anhydrous form of the di-hydrochloride salt of the compound of formula (II) by subjecting the salt to humidity conditions in the range of from about 50% RH to about 100% RH. Such humidity conditions may be achieved by for example, use of salt solutions and/or “wet” nitrogen.

Crystalline Form II-A may be characterized by its XRD spectra which comprises the following peaks:

TABLE 3
Form II-A: Di-hydrate Crystalline Form of the Di-Hydrochloride Salt
of the Compound of Formula (II)

| Pos. [°2Th.] | d-spacing [Å] | Rel. Int. [%] |
|---|---|---|
| 3.67 | 24.1 | 82.6 |
| 5.73 | 15.4 | 19.8 |
| 7.37 | 12.0 | 43.0 |
| 8.77 | 10.1 | 59.1 |
| 12.36 | 7.2 | 31.1 |
| 12.58 | 7.0 | 17.3 |
| 15.88 | 5.6 | 12.3 |
| 16.39 | 5.4 | 11.5 |
| 17.38 | 5.1 | 14.3 |
| 18.57 | 4.8 | 100.0 |
| 18.74 | 4.7 | 48.8 |
| 19.50 | 4.6 | 12.8 |
| 20.60 | 4.3 | 49.9 |
| 21.34 | 4.2 | 79.9 |
| 22.26 | 4.0 | 46.1 |
| 22.67 | 3.9 | 33.5 |
| 23.14 | 3.9 | 44.8 |
| 23.66 | 3.8 | 18.9 |
| 24.33 | 3.7 | 25.4 |
| 24.52 | 3.6 | 29.8 |
| 24.90 | 3.6 | 38.3 |
| 25.51 | 3.5 | 19.9 |
| 25.99 | 3.5 | 31.1 |
| 26.33 | 3.4 | 66.5 |
| 26.76 | 3.3 | 12.8 |
| 27.26 | 3.3 | 13.4 |
| 27.85 | 3.2 | 15.2 |
| 28.25 | 3.2 | 45.2 |
| 28.68 | 3.1 | 23.9 |
| 30.24 | 3.0 | 26.1 |
| 30.58 | 2.9 | 23.0 |
| 31.31 | 2.9 | 28.4 |
| 32.02 | 2.8 | 12.2 |
| 32.56 | 2.8 | 16.6 |
| 33.33 | 2.7 | 12.9 |
| 33.64 | 2.7 | 15.9 |
| 35.71 | 2.5 | 14.5 |
| 37.50 | 2.4 | 10.9 |
| 38.50 | 2.3 | 17.9 |
| 43.32 | 2.1 | 55.4 |
| 44.48 | 2.0 | 31.6 |
| 50.46 | 1.8 | 18.5 |

Crystalline Form II-B of the di-hydrochloride salt of the compound of formula (II) may be prepared by isolating the di-hydrochloride salt directly following de-protection of the compound of formula (XV), as described in Scheme 1, above, wherein the de-protection is carried out by reacting the compound of formula (XV) with anhydrous HCl(g) or HCl in any anhydrous organic solvent. Alternatively, crystalline Form II-B may be prepared by re-crystallizing any of the di-hydrochloride salt forms of the compound of formula (II) from a mixture of methanol and ethyl acetate, wherein the ratio of methanol:ethyl acetate as described above.

Crystalline Form II-B may be characterized by its XRD spectra which comprises the following peaks:

TABLE 4
Form II-B: Anhydrous Crystalline Form of the Di-Hydrochloride Salt
of the Compound of Formula (II)

| Pos. [°2Th.] | d-spacing [Å] | Rel. Int. [%] |
|---|---|---|
| 7.32 | 12.1 | 100.0 |
| 7.85 | 11.3 | 26.4 |
| 9.99 | 8.9 | 60.0 |
| 12.28 | 7.2 | 21.8 |
| 12.97 | 6.8 | 15.3 |
| 14.40 | 6.2 | 33.4 |
| 15.67 | 5.7 | 12.8 |
| 15.98 | 5.6 | 17.5 |
| 17.92 | 5.0 | 12.0 |
| 18.32 | 4.8 | 10.3 |
| 19.26 | 4.6 | 16.9 |
| 20.02 | 4.4 | 22.4 |
| 20.21 | 4.4 | 12.2 |
| 21.24 | 4.2 | 21.0 |
| 23.28 | 3.8 | 51.2 |
| 23.64 | 3.8 | 91.0 |
| 24.30 | 3.7 | 24.9 |
| 24.45 | 3.6 | 24.0 |
| 25.14 | 3.5 | 13.9 |
| 26.75 | 3.3 | 52.1 |
| 30.22 | 3.0 | 25.7 |
| 32.32 | 2.8 | 10.1 |
| 33.49 | 2.7 | 32.6 |

The anhydrous, crystalline form (Form II-B) may be inter-converted to the di-hydrate, crystalline form (Form II-A) by re-slurrying Form II-B in water; by subjecting Form II-B to high humidity conditions, by re-crystallizing Form II-B from a mixture of IPA:water; or by re-crystallizing Form II-B from a mixture of methanol:water:ethyl acetate.

The crystalline forms of the present invention were further characterized by their respective TGA (Thermogravimetric Analysis). Total weight loss was obtained on a PYRIS 1 TGA (Perkin Elmer). The samples (˜2-3 mg) were heated at a scanning rate of 10° C./min between 30 and 250° C. Aluminum pans were used for all samples.

The crystalline forms of the present invention were additionally characterized by their respective DSC (Differential Scanning Calorimeter). Thermal analysis was performed using a Diamond DSC (Perkin Elmer). The samples (˜2-3 mg) were sealed in 50 μL aluminum pans with a single hole (50mm hole). The reference and the sample pans were identical. The sample was scanned from 30 to 250° C. at 10° C./min with a 40 mL/min nitrogen purge.

TGA and DSC traces were measured for crystalline Form I-A and I-B of the compound of formula (I) according to the procedure as described above.

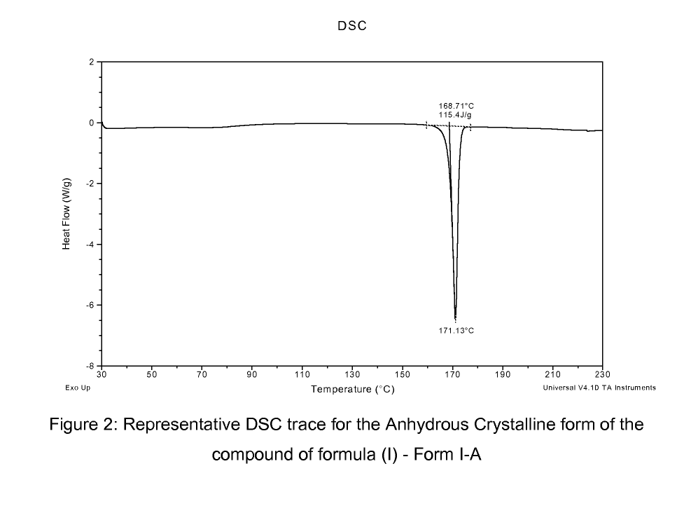

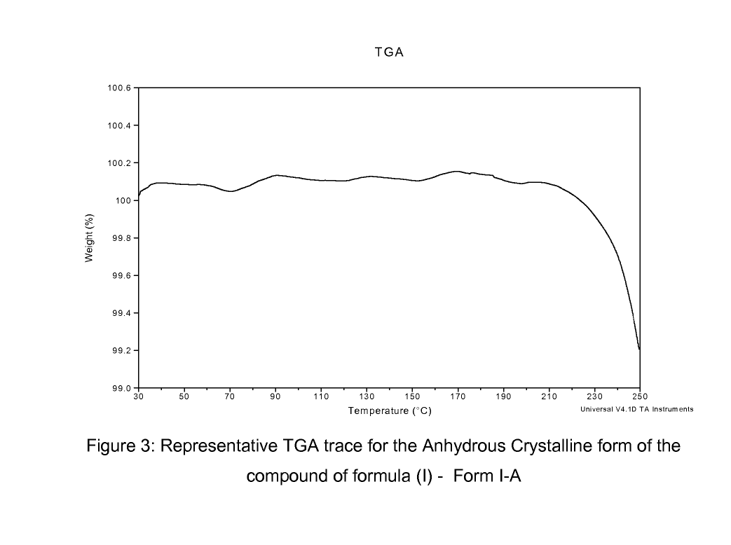

A representative DSC trace for Form I-A (crystalline anhydrous) is shown in FIG. 2. The DSC trace shows a single peak at 171° C. A representative TGA trace for Form I-A (crystalline anhydrous) is shown in FIG. 3. The TGA trace shows an approximately flat trace, indicating no weight loss, indicating that Form I-A is an anhydrous crystalline form.

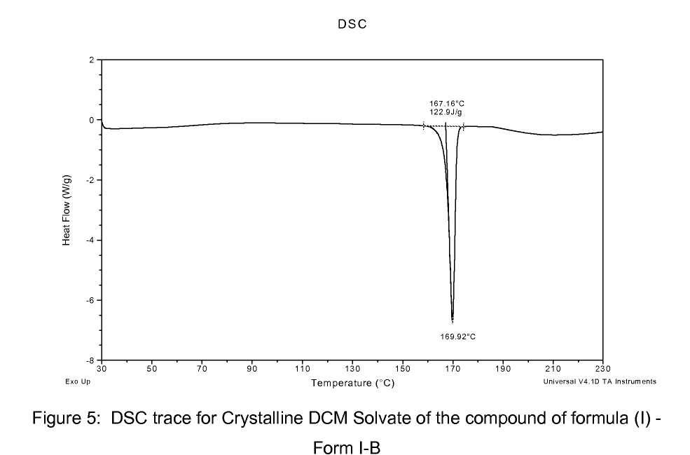

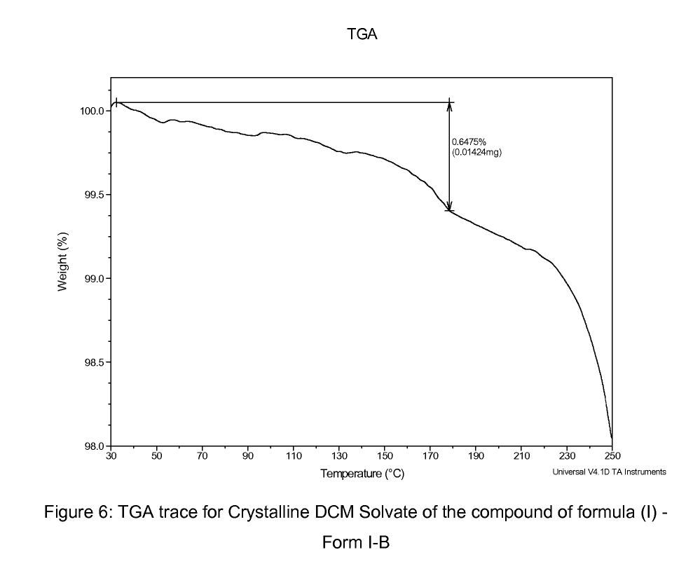

A representative DSC trace for Form I-B (crystalline, DCM solvate) is shown in FIG. 5. The DSC trace shows a single peak at 169° C. A representative TGA trace for Form I-B (crystalline, DCM solvate) is shown in FIG. 6. The TGA trace shows a transition corresponding to a weight loss of 0.65%, indicating that Form I-B is a solvate and that the DCM is not locked within the crystal structure.

TGA traces were measured for crystalline Form II-A and II-B of the di-hydrochloride salt of the compound of formula (II) according to the procedure as described above.

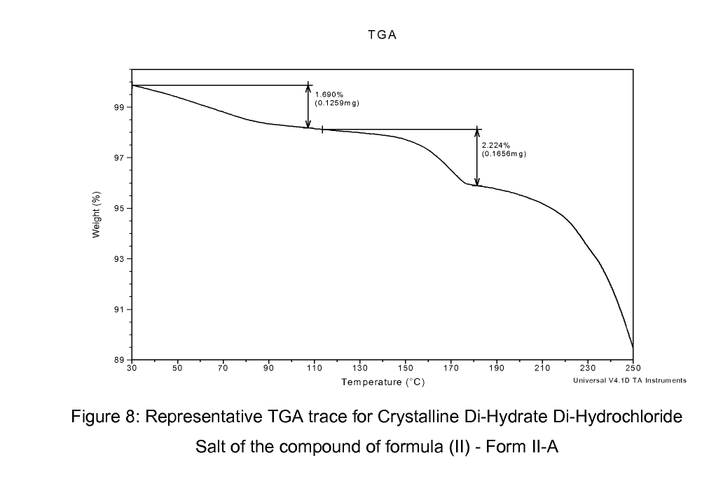

A representative TGA trace for Form II-A (crystalline, di-hydrate) is shown in FIG. 8. The TGA trace shows two transitions, corresponding to a 1.9% weight loss and a 2.2% weight loss, indicating that Form II-A is a di-hydrate crystalline form.

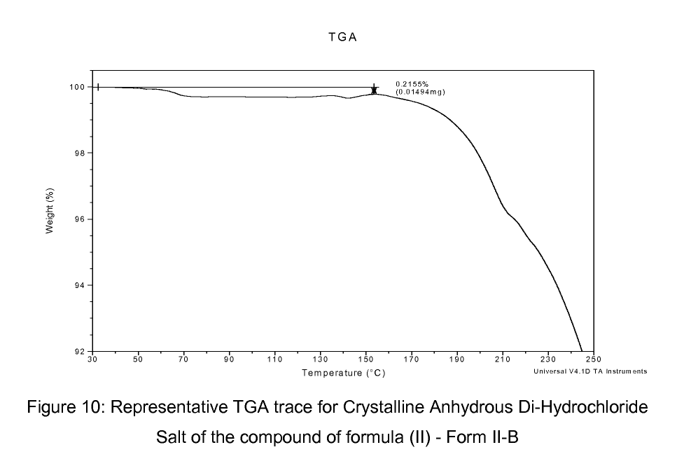

A representative TGA trace for Form II-B (crystalline, anhydrous) is shown in FIG. 10. The TGA trace shows a single weak transition, corresponding to a 0.25% weight loss, indicating that Form II-B is an anhydrous crystalline form.

Pharmaceutical Compositions

In certain pharmaceutical compositions the active ingredient may be present in a micronized form. A micronized form of the compound of Formula (I) has a particle size which allows for going through a sieve of 0.05 inch. Micronization can be performed by art-known methods.

The present invention further comprises pharmaceutical compositions containing a compound of formula (I) or at least one of the pharmaceutically acceptable salts of the compound of formula (II) with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 2 to about 500 mg and may be given at a dosage of from about 0.01 to about 100 mg/kg/day, preferably from about 4 to about 500 mg/ay. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. In particular the compound of Formula (I) may be present in a gastro-retentive sustained release formulation. Said gastro-retentive sustained release formulation allows for a sustained delivery, thus preferably allowing for a once-a-day formulation. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The method of treating disorders described in the present invention may also be carried out using a pharmaceutical composition comprising any of the compounds as defined herein and a pharmaceutically acceptable carrier. The pharmaceutical composition may contain between about 0.01 mg and about 1000 mg, preferably about 2 to about 500 mg, of the compound, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and preferably sustained release formulations), granules, and powders, and liquid forms, such as solutions, syrups, elixers, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

To prepare a pharmaceutical composition of the present invention, a compound of formula (I) or at least one pharmaceutically acceptable salt of the compound of formula (II) as the active ingredient is intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

Compounds of this invention may be administered in any of the foregoing compositions and according to dosage regimens established in the art whenever treatment of disorders as described herein is required.

The daily dosage of the products may be varied over a wide range from about 0.01 to about 1,000 mg per adult human per day. For oral administration, the compositions are preferably provided in the form of tablets containing about 0.01, 0.05, 0.1, 0.5, 1.0, 2.0, 4.0, 8.0, 16.0, 24.0, 36.0, 50.0, 100.0, 150.0, 200.0, 250.0, 300.0, 400.0 and 500.0 milligrams of the active ingredient for the symptomatic adjustment of the dosage to the patient to be treated. An effective amount of the drug is ordinarily supplied at a dosage level of from about 0.01 mg/kg to about 100 mg/kg of body weight per day or any range therein. Preferably, the range is from about 0.01 to about 50 mg/kg of body weight per day, more preferably, from about 0.05 to about 10 mg/kg of body weight per day. The compounds may be administered on a regimen of 1, 2, 3 or 4 times per day, preferably 1 to 2 times per day, more preferably, once a day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

Therapeutically effective dosage levels and dosage regimens for the anti-thrombosis agents disclosed herein, may be readily determined by one of ordinary skill in the art. For example, therapeutic dosage amounts and regimens for pharmaceutical agents approved for sale are publicly available, for example as listed on packaging labels, in standard dosage guidelines, in standard dosage references such as the Physician's Desk Reference (Medical Economics Company or online at http://www.pdrel.com) and other sources.

One skilled in the art will recognize that, both in vivo and in vitro trials using suitable, known and generally accepted cell and/or animal models are predictive of the ability of a test compound to treat or prevent a given disorder. Determination of effective dosages is typically based on animal model studies followed up by human clinical trials and is guided by determining effective dosages and administration protocols that significantly reduce the occurrence or severity of targeted exposure symptoms or conditions in the subject. Suitable models in this regard include, for example, murine, rat, porcine, feline, non-human primate, and other accepted animal model subjects known in the art. Alternatively, effective dosages can be determined using in vitro models (e.g., immunologic and histopathologic assays). Using such models, only ordinary calculations and adjustments are typically required to determine an appropriate concentration and dose to administer a therapeutically effective amount of the biologically active agent(s) (e.g., amounts that are intranasally effective, transdermally effective, intravenously effective, or intramuscularly effective to elicit a desired response).

One skilled in the art will further recognize that human clinical trails including first-in-human, dose ranging and efficacy trials, in healthy patients and/or those suffering from a given disorder, may be completed according to methods well known in the clinical and medical arts.

The following Examples are set forth to aid in the understanding of the invention, and are not intended and should not be construed to limit in any way the invention set forth in the claims which follow thereafter.

In the Examples which follow, some synthesis products may be listed as having been isolated as a residue. It will be understood by one of ordinary skill in the art that the term “residue” does not limit the physical state in which the product was isolated and may include, for example, a solid, an oil, a foam, a gum, a syrup, and the like.

### EXAMPLE 1

### 3-(2,2-Difluoro-2-phenylethylamino)-1-(ethoxycarbonyimethyl)-6-methylpyrazinone

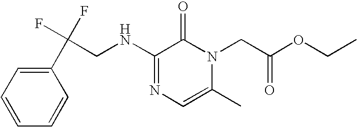

A 2-liter, one-neck round bottom flask (equipped with a nitrogen inlet) was charged with 3-bromo-1-(ethoxycarbonylmethyl)-6-methylpyrazinone (51.5 g, 0.187 mol), 2,2-difluoro-2-phenylethylamine (64.78 g, 0.412 mol), and toluene (450 ml). The reaction mixture was refluxed for 2 days and concentrated to yield a solid. The solid was triturated with ethanol-water (1:1, 890 ml) and the resulting mixture stirred for 2 h. The solid was collected by filtration and then washed with ethanol-water (1:3, 450 ml), filtered, dried and to yield the title compound as a tan solid.

MP: 106-110° C.

### EXAMPLE 2

### 3-(2,2-Difluoro-2-phenylethylamino)-1-(hydroxycarbonylmethyl)-6-methyl-pyrazinone

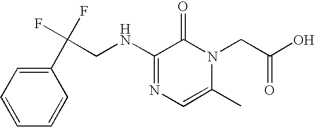

A 3-liter, one neck round bottom flask (equipped with magnetic stirring bar) was charged with 3-(2,2-difluoro-2-phenylethylamino)-1-(ethoxycarbonylmethyl)-6-methylpyrazinone (59.93 g, 0.171 mol), ethanol (1.64 l), water (0.82 l), and 14 M sodium hydroxide (36 ml, 0.504 mol). The reaction mixture was loosely capped and stirred at room temperature for 17 h (overnight). The reaction mixture was concentrated and the aqueous semi-solid residue (˜1000 ml) was diluted with water (360 ml) and acidified with 1 M hydrochloric acid (645 ml). The reaction mixture was stirred for 2 h and the resulting solid was collected by filtration, washed with water (160 ml). The solid was dried in a vacuum oven to yield the title compound as a tan solid.

MP: 65-75° C., decomp

### EXAMPLE 3

### 1-{N-[2-(N′,N″-Bis(tert-butoxycarbonyl)amidinoaminooxy)ethyl]amino}-carbonylmethyl-6-methyl-3-(2,2-difluoro-2-phenethylamino)-pyrazinone

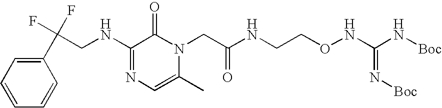

A 5-liter, three-necked round bottom flask (equipped with mechanical stirrer, nitrogen inlet, and an addition funnel) was charged with [N,N′-di(tert-butoxycarbonyl)]-2-aminoethoxyguanidine (80.9 g, 0.228 mol), triethylamine (82 ml, 0.589 mol), and anhydrous N,N-dimethylformamide (1.15 l). In a separate 1-liter, three-necked flask (equipped with magnetic stirring bar) was charged 3-(2,2-difluoro-2-phenylethylamino)-1-(hydroxy-carbonylmethyl)-6-methyl-pyrazinone (61.34 g, 0.190 mol), benzotrazol-1-yloxytris-(dimethylamino)phosphonium hexafluorophosphate (126 g, 0.285 mol, BOP reagent), and anhydrous DMF (600 ml). The resulting mixture was stirred for 15 min then charged into the addition funnel. The mixture was added to the mixture containing the [N,N′-di(tert-butoxycarbonyl)]-2-aminoethoxyguanidine over a 15 min period and the resulting mixture was stirred for 16 h (overnight). The reaction mixture was split into two portions. Each portion was portioned between 2 l of saturated aqueous sodium bicarbonate and 1 l ethyl acetate. The layers were separated and the aqueous extracted with ethyl acetate (2×500 ml). The combined ethyl acetate phases (˜2 l) were washed with water (2×500 ml) and brine (2×500 ml). The workup was repeated on the second portion and the extracts were combined, dried (MgSO₄), and concentrated to yield as residue as a solid. The residue was absorbed onto 300 g of silica gel and chromatographed in two equal batches on a Biotage™ 75 L (800 g silica gel), eluting with hexanes (2 l), ethyl acetate-hexanes 1:3 (4 l), 1:1 (6 4l), 3:2 (4 l), and 3:1 (8 l) to yield the title compound as a yellow foam.

MP: 65-70° C.

Both batches were identical by NMR.

### EXAMPLE 4

1-{N-[2-(amidinoaminooxy)ethyl]amino}carbonylmethyl-6-methyl-3-(2,2-difluoro-2-phenylethylamino)-pyrazinone di-hydrochloride

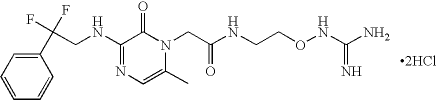

A 250-ml, 4-necked flask (equipped with mechanical stirrer, condenser with nitrogen inlet, thermocouple, and a glass stopper) was charged with 1-{N-[2-(N′, N″-bis(tert-butoxycarbonyl)amidinoaminooxy)ethyl]amino}-carbonyl methyl-6-methyl-3-(2,2-difluoro-2-phenethylamino)-pyrazinone (51 g, 81.8 mol), hydrogen chloride in 1,4-dioxane (4 M, 82 ml, 328 mmol), and anhydrous 1,4-dioxane (100 ml). The reaction mixture was brought to a mild reflux and held there for 15 min. A thick semi-solid formed and was broken up into smaller pieces with a spatula. Stirring was continued at 75° C. (internal) for 1 h, at which time a tan slurry was formed. The reaction was complete as determined by LCMS, the reaction mixture was cooled to room temperature and a tan solid was collected by filtration, washing with anhydrous tetrahydrofuran (200 ml) and anhydrous ethyl ether (200 ml). The filter cake was then digested in absolute ethanol (250 ml) at a mild reflux (ethanol was replenished as needed) for 1 h. The slurry was cooled to room temperature and diluted with diethyl ether (500 ml) and stirred for 15 min. An off-white solid was collected by filtration and washed with diethyl ether (250 ml). The solid was dried to yield the title compound as an off-white solid.

MP: 210-215° C. decomp. gas evolution

Elemental Analysis for C₁₈H₂₅Cl₂F₂N₇O₃ (with 0.03 molar eq ethanol by NMR and 0.1 molar eq water by Karl Fisher titration):

Calculated: % C 43.43,% H 5.12, % N 19.62, % Cl 14.19, % F 7.60

Measured: % C 43.21, % H 5.04, % N 19.50, % Cl 14.45, % F 7.66;

### EXAMPLE 5

### [3-(2,2-Difluoro-2-phenyl-ethylamino)-6-methyl-2-oxo-2H-pyrazin-1-yl]-acetic acid

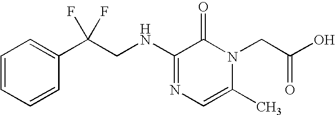

The title compound was prepared according to the procedure shown below and described in details thereinafter.

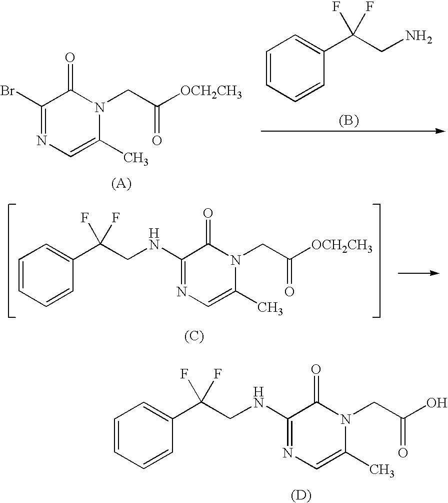

n-Butanol (2919.6 g, 3.6 l), 3-bromo-6-methyl-2-oxo-2(2H-pyrazine acetic acid) ethyl ester (Reagent (A) 600 g, 2.14 mol 1 equiv.)), and 2,2-difluoro-2-phenylethylamine (Reagent (B) 675 g, 4.29 mol, 2.00 equiv.) were charged to the reaction vessel and the reaction mixture heated to reflux. The reaction mixture was maintained at reflux. The reaction was then cooled to 75° C. and a 25% solution of sodium hydroxide (759 g, 600 ml 2.68 equiv.) was added. The reaction mixture was stirred at 75° C. A 22% hydrochloric acid solution (1010 g, 900 ml, 3.13 equiv.) was then added slowly to the reaction mixture. The reaction mixture was allowed to cool to room temperature and then stirred overnight. The next morning the reaction mixture was cooled to 10° C., filtered, the filtrate washed with n-butanol (2.1 l), and then dried at 55° C. to yield the title compound as a residue.

### EXAMPLE 6

### N-[2-[[(aminoiminomethyl)amino]oxy]ethyl]-3-[(2,2-difluoro-2-phenylethyl)amino]-6-methyl-2-oxo-1(2H)-pyrazineacetamide di-hydrochloride salt

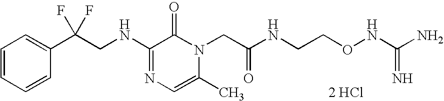

The title compound was prepared according to the procedure shown below and described in detail thereinafter.

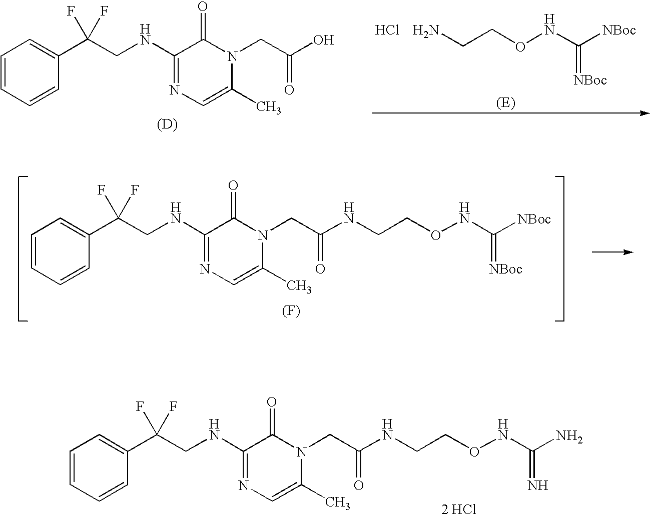

[3-(2,2-Difluoro-2-phenyl-ethylamino)-6-methyl-2-oxo-2H-pyrazin-1-yl]-acetic acid (Reagent (D) 1900 g, 5.57 mol, 1.00 equiv.), 2-chloro-4,6-dimethoxy-1,3-5-triazine (CDMT 1056 g, 5.89 mol, 1.06 equiv.), [N,N′-di(tert-butoxycarbonyl)]-2-aminoethoxyguanidine (Reagent (E) 2240 gm, 6.0 mol, 1.08 equiv.) and tetrahydrofuran (26640 g, 30 l) were charged to a reaction vessel. N-Methyl morpholine (1545.6 g, 1680 ml, 2.72 equiv.) in tetrahydrofuran was slowly added to the suspension and the reaction mixture was stirred. The resulting suspension was then filtered. Anhydrous hydrogen chloride gas (4676.02 g, 128.11 mol, 23 equiv.) was bubbled into the filtrate while maintaining the temperature of the reaction mixture at <40° C. The reaction was stirred at 23° C. overnight and then warmed to 40° C. The reaction mixture was filtered, washed with tetrahydrofuran and then dried at <65° C. and full vacuum overnight to yield the title compound as a residue.

### EXAMPLE 7

### N-[2-[[(aminoiminomethyl)amino]oxy]ethyl]-3-[(2,2-difluoro-2-phenylethyl)amino]-6-methyl-2-oxo-1(2H)-pyrazineacetamide di-hydrochloride salt, di-hydrate

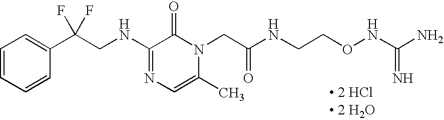

N-[2-[[(aminoiminomethyl)amino]oxy]ethyl]-3-[(2,2-difluoro-2-phenylethyl)amino]-6-methyl-2-oxo-1(2H)-pyrazineacetamide di-hydrochloride salt prepared as in Examples 1 and 2 above (21 kg, 40.19 mol, 1.00 equiv.), water (14.7 kg, 14.7 l, 20.37 equiv.), and methanol (21.59 kg, 27.3 l, 16.8 equiv.) were charged to the reaction vessel and the resulting mixture was heated. The resulting mixture was then cooled to 0-5° C. and filtered. Ethyl acetate (94.71 g, 105 l, 16.8 equiv.) was then slowly added to the filtrate. The resulting suspension was agitated overnight and the filtered. The solid was dried with a slight nitrogen purge under partial vacuum overnight at room temperature to yield the title compound as a residue.

### EXAMPLE 8

### [3-(2,2-Difluoro-2-phenyl-ethylamino)-6-methyl-2-oxo-2H-pyrazin-1-yl]-acetic acid ethyl ester

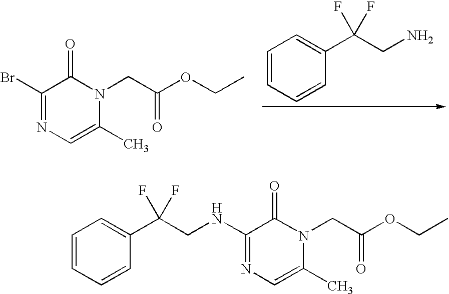

(3-Bromo-6-methyl-2-oxo-2H-pyrazin-1-yl)-acetic acid ethyl ester was reacted with 2,2-difluoro-2-phenyl-ethylamine in the presence of a base, at elevated temperature, to yield the title compound as shown in the Scheme above. Table 5 below lists the reagent amounts, base and temperature combinations applied to this reaction, as well as yield.

TABLE 5

| | | Temp, | Base, | Amine, | Time, | Yield, |
|---|---|---|---|---|---|---|
| Run | Solvent | ° C. | equiv | equiv. | days | area % |
| 1 | toluene | 112 | none | 2.2 | 2 | Complete |
| 2 | toluene | 112 | ⁱPr₂NEt, 1 equiv | 1.1 | 5 | slow |
| 3 | ethanol | 80 | none | 2.2 | 5 | |
| 4 | ethanol | 80 | ⁱPr₂NEt, 1 equiv | 1.1 | 5 | Slow |
| 5 | acetonitrile | 83 | none | 2.1 | 2 | 4% |
| 6 | acetonitrile | 83 | DBU, 1 equiv | 1 | 2 | No rxn |
| 7 | acetonitrile | 83 | DMAP, 1 equiv | 1 | 2 | No rxn |
| 8 | acetonitrile | 83 | NMM, 1 equiv | 1 | 2 | No rxn |
| 9 | acetonitrile | 83 | ⁱPr₂NEt, 1 equiv | 1 | 2 | No rxn |
| 10 | NMP | 120 | none | 2.1 | 1 | 32% |
| 11 | NMP | 120 | DBU, 1 equiv | 1 | 1 | 16% |
| 12 | NMP | 120 | DMAP, 1 equiv. | 1 | 1 | 2% |
| 13 | NMP | 120 | NMM, 1 equiv | 1 | 1 | 10% |
| 14 | NMP | 120 | ⁱPr₂NEt, 1 equiv | 1 | 1 | 10% |
| 15 | NMP | 140 | none | 2.1 | 1 | 85% |
| 16 | NMP | 140 | DBU, 1 equiv | 1 | 1 | 49% |
| 17 | n-butanol | 118 | none | 2 | 6 hours | w/o isolation |

### EXAMPLE 9

### 1-{N-[2-(N′,N″-Bis(tert-butoxycarbonyl)amidinoaminooxy)ethyl]amino}-carbonylmethyl-6-methyl-3-(2,2-difluoro-2-phenethylamino)-pyrazinone

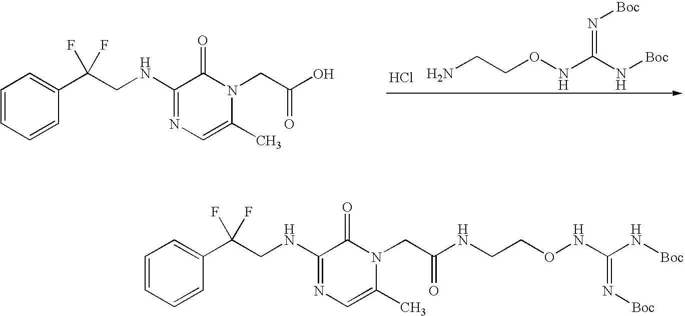

[3-(2,2-Difluoro-2-phenyl-ethylamino)-6-methyl-2-oxo-2H-pyrazin-1-yl]-acetic acid was reacted with [N,N′-di(tert-butoxycarbonyl)]-2-aminoethoxyguanidine in the presence of a coupling agent and base, to yield the title compound as shown in the Scheme above. Table 6 below lists the reagent amounts, base and temperature combinations applied to this reaction, as well as yield.

TABLE 6

| | Coupling Agent/ | | |
|---|---|---|---|
| Run | Base | Solvent | Results & Conclusions |
| 1 | IBCF/NMM | THF | Complete reaction; |
| | | | Two impurities which could not be |
| | | | removed without chromatography |
| 3 | EDCI/HOBt/Et₃N | CH₃CN | <3% starting material evident from |
| | | | 1-3 hours |
| 4 | Cyanuric | THF | Reaction incomplete |
| | Chloride/NMM | | |
| 5 | CDMT/NMM | THF | Solution yield >95% wt; |
| | | | acceptable purity |

### EXAMPLE 10

### (N-[2-[[(aminoiminomethyl)amino]oxy]ethyl]-3-[(2,2-difluoro-2-phenylethyl)amino]-6-methyl-2-oxo-1(2H)-pyrazineacetamide)

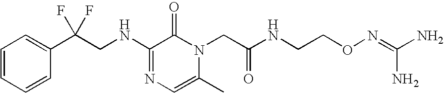 A: Title Compound as Plates:

N-[2-[[(aminoiminomethyl)amino]oxy]ethyl]-3-[(2,2-difluoro-2-phenylethyl)amino]-6-methyl-2-oxo-1(2H)-pyrazineacetamide di-hydrochloride salt (99.2 g) was dissolved in water (3700 mL) and then treated with 1 N NaOH (400 mL) at ambient temperature. The title compound precipated out immediately after adding the NaOH. The while solid was isolated by filtration.

B: Title Compound as Needles:

N-[2-[[(aminoiminomethyl)amino]oxy]ethyl]-3-[(2,2-difluoro-2-phenylethyl)amino]-6-methyl-2-oxo-1(2H)-pyrazineacetamide di-hydrochloride salt dihydrate was suspended in methanol with 2.0 equivalents of 5N sodium hydroxide at room temperature. Water was added to dissolve the resultant sodium chloride. The suspension was heated to ˜60° C., followed by a polish filtration. The filtrate was re-heated to ˜60° C. and additional water was added. The solution was maintained at ˜60° C. The solution was cooled with stirring to ˜55° C. and seeded, followed by a stepwise cooling procedure until the final temperature reached ˜25° C. The title compound, as needles was then filtered, washed and dried under house vacuum at 45-50° C. overnight.

### EXAMPLE 11

### (N-[2-[[(aminoiminomethyl)amino]oxy]ethyl]-3-[(2,2-difluoro-2-phenylethyl)amino]-6-methyl-2-oxo-1(2H)-pyrazineacetamide)

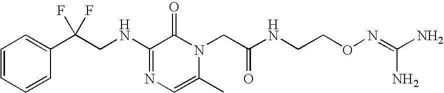

The following is a recipe/procedure for the synthesis of (N-[2-[[(aminoiminomethyl)amino]oxy]ethyl]-3-[(2,2-difluoro-2-phenylethyl)amino]-6-methyl-2-oxo-1(2H)-pyrazineacetamide).

Several batches of the anhydrous crystalline form of the compound of formula (I) (the title compound) were made using the recipe as described below.

STEP A: [3-(2,2-Difluoro-2-phenyl-ethylamino)-6-methyl-2-oxo-2H-pyrazin-1-yl]-acetic acid

A 4-neck round bottom flask, equipped with thermometer, and addition funnel, is charged with 3-bromo-6-methyl-2-oxo-2H-pyrazin-1-yl)-acetic acid ethyl ester (44.00 g, 160.0 mmol), anhydrous Na₂HPO₄ (31.8 g, 224 mmol), and n-butanol (210 g). 2,2-Difluoro-2-phenyl-ethylamine (30.15 g, 192 mmol) is then added in one portion. The resulting suspension is heated to reflux over 30-90 min and further stirred for 12-16 h. The suspension is cooled to 75-90° C. over 30-60 min, and water (130 g) is added over 15-30 min, resulting in a clear 2-phasic reaction mixture. 30% NaOH(aq) ( 95.2 g, 714 mmol) is added and the reaction mixture is further stirred for 60 min at 75-85° C. After completion of the hydrolysis, 32/34% HCl_{(aq)} (85 g, 778 mmol) is added dropwise, resulting in the precipitation of the product. Complete precipitation is achieved by cooling the suspension to 0-10° C. and stirring at this temperature for additional 30 min. The product ([3-(2,2-Difluoro-2-phenyl-ethylamino)-6-methyl-2-oxo-2H-pyrazin-1-yl]-acetic acid) is centrifuged and washed with water (100 g) and then with a mixture of ethanol (40 g) and water (50 g). The isolated product is then vacuum dried at 40-60° C.

Note: Step A may alternatively be completed by reacting the 3-bromo-6-methyl-2-oxo-2H-pyrazin-1-yl)-acetic acid ethyl ester with the HCl salt of 2,2-difluoro-2-phenyl-ethylamine. In this case, an aqueous work-up with TBME and 30% NaOH_{(aq)} is required to isolate the product.

STEP B: 1-{N-[2-(N′,N″-Bis(tert-butoxycarbonyl)amidinoaminooxy)ethyl]amino}-carbonylmethyl-6-methyl-3-(2,2-difluoro-2-phenethylamino)-pyrazinone

A 3-neck round bottom flask, equipped with thermometer, pH-electrode, and bubbler, is charged with [3-(2,2-Difluoro-2-phenyl-ethylamino)-6-methyl-2-oxo-2H-pyrazin-1-yl]-acetic acid(20.00 g, 61.9 mmol), [N,N′-di(tert-butoxycarbonyl)]-2-aminoethoxyguanidine (21.68 g, 68.1 mmol), 1-hydroxy-benzotriazol (0.42 g, 3.09 mmol), acetonitrile (140 g), and water (50 g). The resulting solution should have a pH of 5.6-5.9. If not the pH is adjusted by addition of 4-methylmorpholine or 1 N HCl_{(aq)}. A 1M solution of N,N′-dicyclohexyl-carbodiimide (DCC) in acetonitrile (65 ml, 65 mmol) is then added over 30-45 min at room temperature, while the pH of the mixture is maintained at about pH 5.5-6.1. After an additional 90-120 min stirring, the resulting suspension is cooled to 0-10° C. and stirred for 60 min. N,N′-Dicyclohexylurea (DCU) is removed by filtration and washed with a mixture of acetonitrile (16 g) and water (5 g). About 160 g of the solvent is removed by distillation (200 mbar, 40-50° C.) and replaced with methanol (180 g). After stirring the resulting mixture at 50° C. for 10-15 min, an additional about 40 g of solvent is removed and water (80 g) is added. After stirring 5-15 min at 50° C. the solution is cooled to 40° C. and seeded. The resulting suspension is stirred for 60 min, cooled to 0-5° C. over 2-4 h and further stirred at least 2 h. The product (1-{N-[2-(N′,N″-Bis(tert-butoxycarbonyl)amidinoaminooxy)ethyl]amino}-carbonylmethyl-6-methyl-3-(2,2-difluoro-2-phenethylamino)-pyrazinone) is centrifuged and washed with a cold mixture of methanol (40 g) and water (50 g). The isolated product is then vacuum dried at 35-45° C.

Note: Step B above may alternatively be completed using the HCl salt of [N,N′-di(tert-butoxycarbonyl)]-2-aminoethoxyguanidine. The use of the corresponding HCl salt results in the need to adjust the reaction mixture pH to about 5.6-5.9 by the addition of 1 equivalent of 4-methylmorpholin.

STEP C: N-[2-[[(aminoiminomethyl)amino]oxy]ethyl]-3-[(2,2-difluoro-2-phenylethyl)amino]-6-methyl-2-oxo-1(2H)-pyrazineacetamide

A 3-neck round bottom flask, equipped with thermometer, pH-electrode, and bubbler, is charged with 20% HCl_{(aq)} (65.7 g, 360 mmol). To this solution are added, in portions, 1-{N-[2-(N′,N″-Bis(tert-butoxycarbonyl)amidinoaminooxy)ethyl]amino}-carbonylmethyl-6-methyl-3-(2,2-difluoro-2-phenethylamino)-pyrazinone (15.0 g, 24.0 mmol) at room temperature (23-27° C.) over 15 min. After completion of the reaction (stirring for 2-4 h), 15% Na₂CO_{3(aq)} (109.7 g, 156 mmol) is added, adjusting the pH to about 1.7-2.0. Water (25.0 g) is then added and the resulting suspension heated to about 30-40° C. Activated carbon Norit CN1 is added (1.5 g), the resulting suspension is stirred for 30-90 min and filtered over Celite®. The Celite® is washed with water (25 g). The pH of the filtrate is adjusted to about 3.5 by addition of 15% Na₂CO_{3(aq)} (6.9 g, 9 mmol). After seeding, the resulting mixture is stirred at room temperature for 30-60 min. The product is then completely precipitated by addition of 15% Na₂CO_{3(aq)} (31.8 g, 45 mmol) with the resulting mixture reaching a pH 8.5. The resulting thick suspension is cooled to 0-5° C. and further stirred at least for 60 min. Product (N-[2-[[(aminoiminomethyl)amino]oxy]ethyl]-3-[(2,2-difluoro-2-phenylethyl)amino]-6-methyl-2-oxo-1(2H)-pyrazineacetamide) is centrifugated and washed with cold water (20 g). The product is isolated as a solid.

STEP D: Recrystallisation of (N-[2-[[(aminoiminomethyl)amino]oxy]ethyl]-3-[(2,2-difluoro-2-phenylethyl)amino]-6-methyl-2-oxo-1(2H)-pyrazineacetamide)

A mixture of the wet product (22-25 g), prepared as in Step C above, and methanol (60 g) is stirred at 50-60° C. till complete dissolution. Water (15 g) is added and the solution cooled to 40° C. After seeding and stirring for 120-180 min, additional water (20 g) is slowly added and the suspension is cooled to 0-5° C. over 3 h. After stirring for 1-3 h, the product is centrifuged and washed with a cold mixture of water (10 g) and methanol (10 g). The isolated product is then vacuum dried at 35° C.

### EXAMPLE 12

### Solubility of the Compound of Formula (I)

The solubility of the compound of formula (I) was measured as a function of time in water, 0.1N HCl, 0.1N NaOH, citrate buffers at pH 2, 4 and 6, and borate buffers at pH 8 and 10, and simulated intestinal fluid (SIF).

More specifically, an excess of the compound of formula (I) was added to the selected solvent. The resulting mixtures were agitated in a 25° C. water bath. The sample vials were pulled out at each time point, the pH was measured and the sample was filtered a 0.45 μm PTFE membrane filter. The filtrate was appropriately diluted with the dilution solvent and the concentration of the compound was measured using HPLC. HPLC conditions were as follows: Method: Isocratic method, 70% mobile phase A −30% mobile phase B; Column: Supelco Discovery HS-C18, 3 μm, 4.6×150mm; Mobile Phase A: 0.05% TFA in water; B: 0.05% TFA in acetonitrile; Flow rate: 1.0 mL/min; Detection: UV: Wavelength: 240 nm, bandwidth 4 nm; Reference wavelength: 450 nm, bandwidth 80 nm; Run time: 6 min; Injection volume: 10 μL.

Measured solubility as a function of time for the listed solvents was as listed in Table 7, below.

TABLE 7

| | Solubility (mg/mL) | | | | | | |
|---|---|---|---|---|---|---|---|
| Solvent | ½ hr | 1 hr | 2 hr | 4 hr | 24 hr | 48 hr | 7 days |
| 0.1N HCl | 31.6 | 40.2 | 40.9 | 43.0 | 40.4 | 39.2 | 26.9* |
| pH 2 | 7.6 | 8.7 | 8.8 | 10.4 | 6.9 | 7.5 | 4.0* |
| pH 4 | 2.1 | 2.4 | 2.8 | 2.5 | 2.7 | 2.7 | ND |
| pH 6 | 2.1 | 1.8 | 1.8 | 2.0 | 1.6 | 1.5 | 1.3* |
| pH 8 | ND | ND | ND | 1.3 | 1.2 | 1.2 | ND |
| pH 10 | ND | ND | ND | 1.0 | 1.0 | 1.0 | ND |
| water (pH ˜9) | ND | ND | ND | 1.1 | 1.1 | 1.1 | ND |
| SIF (pH ˜6.75) | ND | ND | ND | 2.8 | 2.7 | 2.7 | ND |
| 0.1N NaOH | 0.92 | 0.98 | 0.98 | 1.0 | 1.0 | 0.99 | ND |
| (pH ˜12.8) | | | | | | | |

*7 day samples were kept at room temperature with no agitation;
ND = Not determined

The measured solubility of the compound of formula (I) was stable as a function of time in water, simulated intestinal fluid, 0.1 N NaOH and borate buffer at pH 8 and pH 10. The solubility of the compound of formula (I) was greatest in SIF (˜2.7-2.8 mg/mL) and decreased with increased pH. The XRD patterns for the solids collected from these samples matched the XRD pattern of the initial sample of the compound of formula (I).

### EXAMPLE 13

### PROPHETIC EXAMPLE

As a specific embodiment of an oral composition, 100 mg of the compound of formula (I) is formulated with sufficient finely divided lactose to provide a total amount of 580 to 590 mg to fill a size O hard gel capsule.

### EXAMPLE 14

A gastro-retentive sustained release formulation of the compound of Formula (I) contained 40% (w/w) of the micronized compound of Formula (I), 48.5 % (w/w) of microcrystalline cellulose, 10% (w/w) Methocel K100 LV, 0.75% (w/w) of butylated hydroxytoluene, 1% (w/w) magnesium stearate. Said mixture was processed into a slug of 500 mg which was inserted into a capsule.

Another gastro-retentive sustained release formulation of the compound of Formula (I) contained 40% (w/w) of the micronized compound of Formula (I), 18.25% (w/w) of microcrystalline cellulose, 40% Methocel L100 LV, 0.75% (w/w) of butylated hydroxytoluene, 1% (w/w) magnesium stearate. Said mixture was processed into a slug of 500 mg which was inserted into a capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

We Claim:

1. A compound of formula (I)

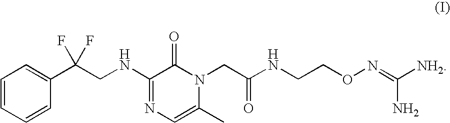

2. The compound of claim 1, wherein the compound of formula (I) is substantially pure.

3. The compound of claim 1, wherein the compound of formula (I) is substantially free of any corresponding salt form.

4. The compound of claim 1, wherein the compound of formula (I) is in and isolated form.

5. A pharmaceutical composition comprising a pharmaceutically acceptable carrier and the compound of claim 1.

6. A pharmaceutical composition made by mixing the compound of claim 1 and a pharmaceutically acceptable carrier.

7. A process for making a pharmaceutical composition comprising mixing the compound of claim 1 and a pharmaceutically acceptable carrier.

8. A methods of inhibiting a serine protease comprising administering to a subject in need thereof a therapeutically effective amount of the compound of claim 1.

9. The method of claim 8, wherein the serine protease is selected from the group consisting of leukocyte neutrophil clastase, chymotrypsin, trypsin, pancreaatic clastase, cathepsin G, thrombin, factor Xa, thermolysin and pepsin.

10. The method of claim 8, wherein the serine protease is thrombin.

11. A method of treating aberrant proteolysis due to a serine protease comprising administering to a subject in need thereof a therapeutically effective amount of the compound of claim 1.

12. A method of reducing blood coagulation comprising administering to a subject in need thereof a therapeutically effective amount of the compound of claim 1.

13. A method of treating thrombosis comprising administering to a subject in need thereof a therapeutically effective amount of the compound of claim 1.

14. The method of claim 13, wherein the thrombosis is associated with or a result of one or more of ischemia, viral infection, stroke, restenosis, myocardial infarction, complication associated with artificial heart valves, complication associated with a stent, arterial fibrillation, uneven blood flow or pooling, disseminated intramuscular coagulopathy which occurs during septic shock, unstable angina, disseminated intramuscular coagulation caused by trauma, coronary artery bypass, hip replacement, thrombolytic therapy, sepsis, hemodialysis, adult respiratory distress syndrome, rheumatoid arthritis, ulcerative colitis, induration, metastasis, hypercoagulability during chemotherapy, fibrin formation in the eye and wound healing.

15. The method of claim 13, wherein the thrombosis is thrombosis associated with ischemia, restenosis, myocardial infarction, coronary artery bypass, hip replacement, thrombolytic therapy or wound healing.

16. A method of treating arterial or venous thrombosis comprising administering to a subject in need thereof, a therapeutically effective amount of the compound of claim 1.

17. A crystalline form of the compound of formula (I)

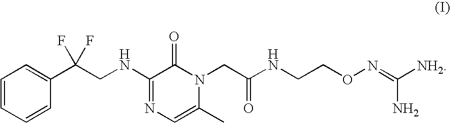

18. The crystalline form of claim 17, wherein the crystalline form is anhydrous and non-hygroscopic.

19. The anhydrous crystalline form of claim 18, wherein the crystalline form is needle-like in appearance.

21. A crystalline, anhydrous form of the compound of formula (I) 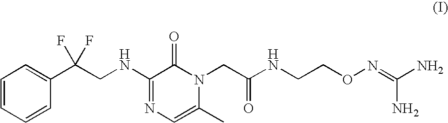
comprising the following X-ray diffraction peaks:

| Pos. [°2Th.] | d-spacing [Å] |
|---|---|
| 4.46 | 19.8 |
| 7.64 | 11.6 |
| 7.97 | 11.1 |
| 8.72 | 10.1 |
| 9.16 | 9.7 |
| 15.53 | 5.7 |
| 15.97 | 5.6 |
| 16.73 | 5.3 |
| 20.11 | 4.4 |

22. A crystalline, DCM solvate form of the compound of formula (I)

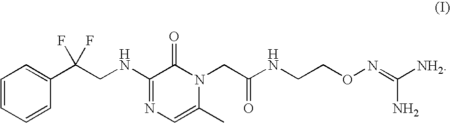

23. A crystalline, DCM solvate form of the compound of formula (I) 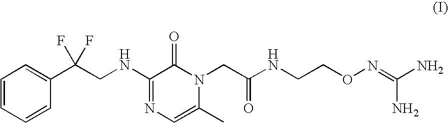
comprising the following X-ray diffraction peaks:

| Pos. [°2Th.] | d-spacing [Å] |
|---|---|
| 4.47 | 19.8 |
| 7.65 | 11.6 |
| 7.95 | 11.1 |
| 8.71 | 10.2 |
| 9.13 | 9.7 |
| 15.52 | 5.7 |
| 15.97 | 5.6 |
| 16.74 | 5.3 |

24. A di-hydrochloride salt of the compound of formula (II)

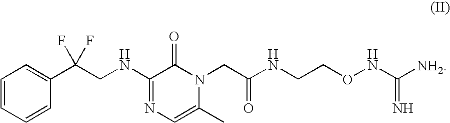

25. A di-hydrochloride salt as in claim 24, wherein the di-hydrochloride salt is crystalline and di-hydrate.

26. A di-hydrochloride salt as in claim 24, wherein the di-hydrochloride salt is crystalline and anhydrous.

27. A crystalline, di-hydrate form of the di-hydrochloride salt of compound of formula (II) 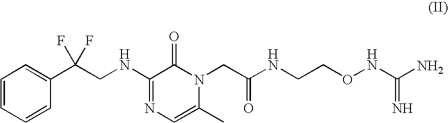
comprising the following X-ray diffraction peaks:

| Pos. [°2Th.] | d-spacing [Å] |
|---|---|
| 3.67 | 24.1 |
| 5.73 | 15.4 |
| 7.37 | 12.0 |
| 8.77 | 10.1 |
| 12.36 | 7.2 |
| 12.58 | 7.0 |
| 15.88 | 5.6 |
| 16.39 | 5.4 |
| 17.38 | 5.1 |
| 18.57 | 4.8 |
| 18.74 | 4.7 |
| 19.50 | 4.6 |
| 20.60 | 4.3 |
| 21.34 | 4.2 |
| 22.26 | 4.0 |
| 22.67 | 3.9 |
| 23.14 | 3.9 |
| 23.66 | 3.8 |
| 24.33 | 3.7 |
| 24.52 | 3.6 |
| 24.90 | 3.6 |
| 25.51 | 3.5 |
| 25.99 | 3.5 |
| 26.33 | 3.4 |
| 26.76 | 3.3 |
| 27.26 | 3.3 |
| 27.85 | 3.2 |
| 28.25 | 3.2 |
| 28.68 | 3.1 |
| 30.24 | 3.0 |
| 30.58 | 2.9 |
| 31.31 | 2.9 |
| 32.02 | 2.8 |
| 32.56 | 2.8 |
| 33.33 | 2.7 |
| 33.64 | 2.7 |
| 35.71 | 2.5 |
| 37.50 | 2.4 |
| 38.50 | 2.3 |
| 43.32 | 2.1 |
| 44.48 | 2.0 |
| 50.46 | 1.8 |

28. A crystalline, anhydrous form of the di-hydrochloride salt of compound of formula (II) 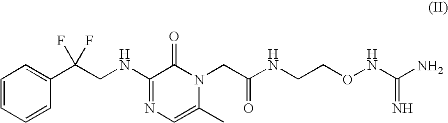
comprising the following X-ray diffraction peaks:

| Pos. [°2Th.] | d-spacing [Å] |
|---|---|
| 7.32 | 12.1 |
| 7.85 | 11.3 |
| 9.99 | 8.9 |
| 12.28 | 7.2 |
| 12.97 | 6.8 |
| 14.40 | 6.2 |
| 15.67 | 5.7 |
| 15.98 | 5.6 |
| 17.92 | 5.0 |
| 18.32 | 4.8 |
| 19.26 | 4.6 |
| 20.02 | 4.4 |
| 20.21 | 4.4 |
| 21.24 | 4.2 |
| 23.28 | 3.8 |
| 23.64 | 3.8 |
| 24.30 | 3.7 |
| 24.45 | 3.6 |
| 25.14 | 3.5 |
| 26.75 | 3.3 |
| 30.22 | 3.0 |
| 32.32 | 2.8 |
| 33.49 | 2.7 |

29. A process for the preparation of a compound of formula (I) 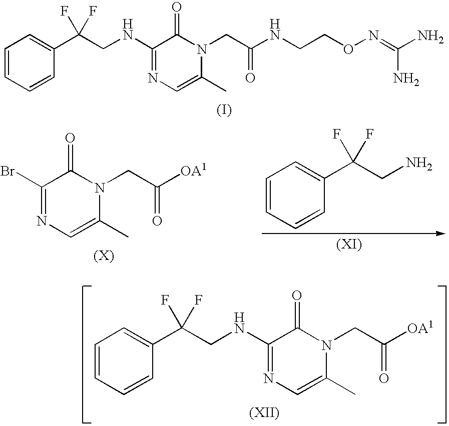
comprising reacting a compound of formula (X), wherein A¹ is a lower alkyl, with a compound of formula (XI) or its corresponding HCl salt, in an organic solvent, to yield the corresponding compound of formula (XII); 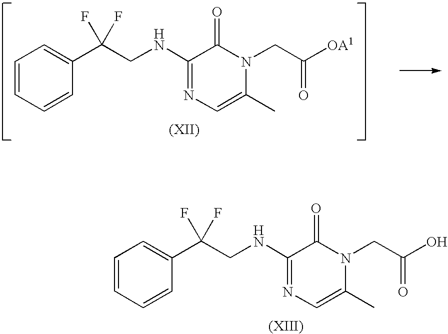
saponifying the compound of formula (XII) with an aqueous base, to yield the corresponding compound of formula (XIII); 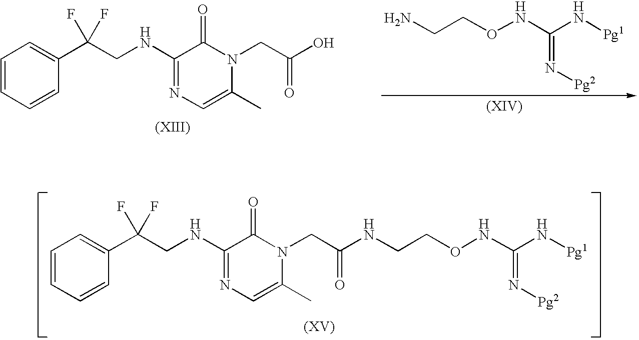
reacting the compound of formula (XIII) with a compound of formula (XIV) or its corresponding HCl salt, wherein Pg¹ and pg² are nitrogen protecting groups, in the presence of a coupling agent, in an organic solvent, to yield the corresponding compound of formula (XV); 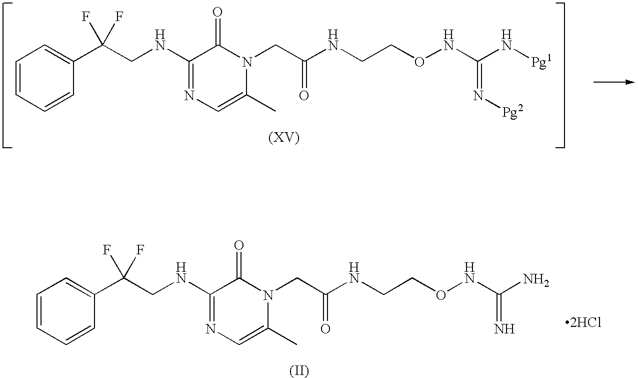
reacting the compound of formula (XV) with HCl, to yield the corresponding di-hydrochloride salt of the compound of formula (II); 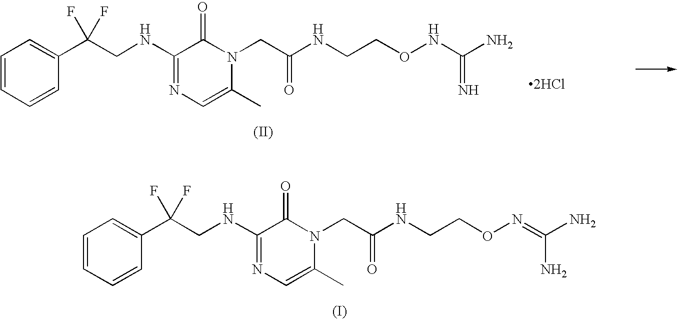
reacting the di-hydrochloride salt of the compound of formula (II) with a base, in water or a mixture of solvents, to yield the corresponding compound of formula (I).

30. A process as in claim 29 wherein the compound of formula (XI) is present in an amount in the range of from about 1.2 to about 2.2. molar equivalent.

31. A process as in claim 29, wherein the compound of formula (X) is reacted with a compound of formula (XI) in the presence of an inorganic base which does not liberate water.

32. A process as in claim 31, wherein the inorganic base isn NaHPO4 and wherein the NaHPO4 is present in about 1.4 molar equivalents.

33. A process as in claim 29, wherein the organic solvent is butanol and wherein the compound of formula (X) is reacted with the compound of formula (XI) at about reflux temperature.

34. A process as in claim 29, wherein the compound of formula (XII) is reacted with aqueous NaOH.

35. A process as in claim 29, wherein Pg¹ and Pg² are each t-butoxycarbonyl and wherein the compound of formula (XIV) is present in an amount in the range of from about 1.05 to about 2.0 molar equivalents.

36. A process as in claim 29, wherein the coupling agent is CDMT or a DCC/HOBt mixture wherein the HOBt is present in an about 5 mole %.

37. A process as in claim 29, wherein the compound of formula (XIII) is reacted with the compound of formula (XIV) or its corresponding HCl salt in acetonitrile and water.

38. A process as in claim 29, wherein the compound of formula (XV) is reacted with aqueous HCl, in an organic solvent.

39. A process as in claim 29, wherein the di hydrochloride salt of the compound of formula (II) is reacted with an excess amount of Na₂CO₃ in water.

40. A process as in claim 29, wherein the di hydrochloride salt of the compound of formula (II) is not isolated.

41. A process for the preparation of a compound of formula (I) 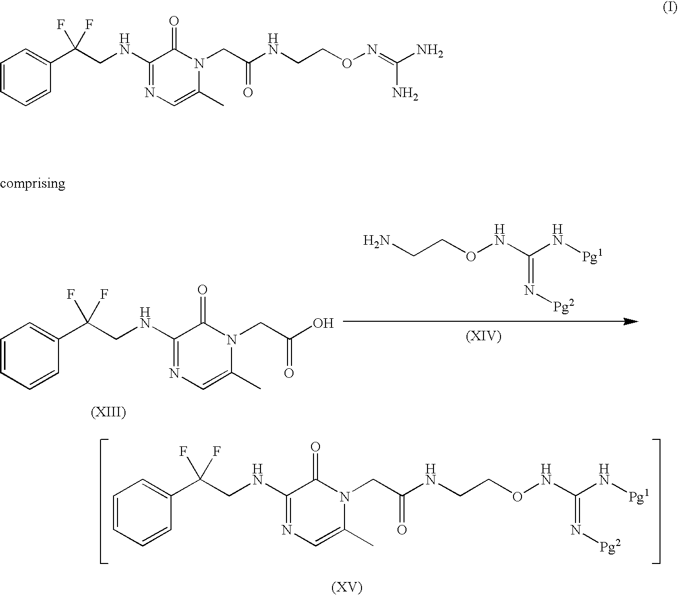
reacting a compound of formula (XIII) with a compound of formula (XIV) or its corresponding HCl salt, wherein Pg¹ and Pg² are nitrogen protecting groups, in the presence of a coupling agent, in an organic solvent, to yield the corresponding compound of formula (XV); which is not isolated; 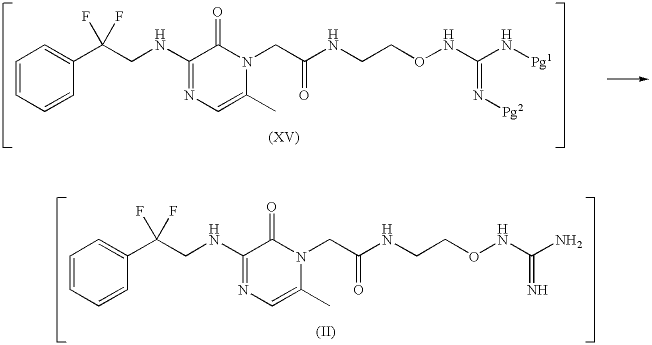
reacting the compound of formula (XV) with an acid, to yield the corresponding compound of formula (II) as its corresponding acid addition salt; which is not isolated; 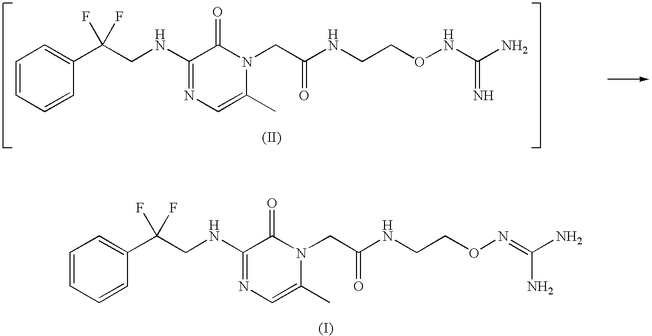
reacting the compound of formula (II) as its corresponding acid addition salt with a base, in water, to yield the corresponding compound of formula (I).

42. A process as in claim 41, wherein the acid is aqueous HCl and wherein the base is Na₂CO₃.

43. A product prepared according to the process of claim 29.

44. A product prepared according to the process of claim 41.

45. A process for the re-crystallization of the compound of formula (I) comprising the steps of (a) dissolving the compound of formula (I) is a mixture of about 80:about 20 methanol:water;
(b) heating the mixture of step (a);
(c) filtering mixture of step (b);
(d) adding water to the mixture of step (c) to a final solvent ratio of about 33 about 67 methanol:water;
(e) cooling the mixture of step (d), which results in the precipitation of substantially pure compound of formula (I); and
isolating the precipitate of substantially pure compound of formula (I) by filtration.
